(19) United States
(12) Patent Application Publication
(10) Pub. No.: US 2011/0184389 A1
(43) Pub. Date: Jul. 28, 2011

(54) OSMOTIC PUMP APPARATUS AND ASSOCIATED METHODS

(75) Inventors: Eric A. Grovender, Minneapolis, MN (US); Ashok V. Joshi, Salt Lake City, UT (US); John Howard Gordon, Salt Lake City, UT (US); Sai Bhavaraju, West Jordan, UT (US); William P. Van Antwerp, Valencia, CA (US)

(73) Assignee: MEDTRONIC, INC., Minneapolis, MN (US)

(21) Appl. No.: 13/077,655

(22) Filed: Mar. 31, 2011

Related U.S. Application Data
(62) Division of application No. 11/591,374, filed on Nov. 1, 2006.

(51) Int. Cl. A61M 37/00

(52) U.S. Cl. 604/891.1; 604/892.1

(57) ABSTRACT

Apparatuses and methods for pumping fluids such as fluid medications are disclosed. Embodiments of the invention provide an osmotic pump fluid delivery apparatus including elements designed to control the fluid delivery rate. Typical embodiments of the invention include an arrangement of elements such as solute reservoirs that can manipulate the solute concentrations within an inner osmotic compartment or compartments of an osmotic pump so as to control fluid delivery from the pump. Other embodiments include sealed electro-osmotic pumps that do not discharge ions into the surroundings or require water from an external source. These embodiments of the invention provide new ways to control fluid delivery in apparatuses that employ osmotic processes to function.

### CROSS REFERENCE TO RELATED APPLICATIONS

This application is a Divisional Application claiming priority under Section 120 to U.S. patent application Ser. No. 11/591,374, filed Nov. 1, 2006, the contents of which are incorporated herein by reference.

### BACKGROUND OF THE INVENTION

1. Field of the Invention

This invention relates generally to osmotic pump apparatuses and associated methods for delivering fluids such as fluid medications.

2. Description of the Related Art

In a number pathological conditions, it is desirable to deliver fluids such as fluid medications gradually over a period of time. A common apparatus for the gradual administration of fluids into the human body is an intravenous administration set, one in which gravity induced hydrostatic infusion dispenses a fluid from a suspended bottle or bag above the patient. Additional devices and methods for the gradual administration of fluids have been devised for example to provide patients with greater mobility and include for example devices that utilize osmosis for fluid delivery. Osmosis is the transfer of a solvent, e.g., water, across a barrier, generally from an area of lesser solute concentration to an area of greater solute concentration. A variety of osmotic and electro-osmotic pumps that utilize osmosis and electro-osmosis to deliver a fluid are well known in the art. Osmotic and electro-osmotic pumps described in the art typically include one or more osmotic compartments that are adapted for the osmotic processes that drive fluid delivery.

One common type of osmotic fluid delivery device is an electro-osmotic cell coupled with a delivery pump. Such electro-osmotic pumps typically operate by utilizing an electrochemical cell in combination with an ion-selective membrane to create a driving force for fluid delivery. Generally, two types of osmotic transport are simultaneously occurring within an operating electro-osmotic cell. A first type of osmosis is electro-osmosis, whereby charged ions are driven across an ion-exchange membrane as the cell is operated, thereby dragging water molecules along with them. A second type of transport is osmosis due to environmental conditions. Electro-osmotic pumps typically include an electric controller as part of an electrical circuit that when completed, causes electrode reactions to occur. In an illustrative reaction, water is extracted from a first electrode cell and ultimately driven across an ion-exchange membrane into a second electrode cell. The water moves a displaceable member which in turn displaces the fluid held in a fluid reservoir such as a fluid medication reservoir. In medication delivery devices for example, the medication delivery rate can then be controlled by the magnitude of current output from the electrical controller.

During operation of osmotic and electro-osmotic fluid delivery devices, the relative concentrations of salts within the osmotic compartments change, causing significant changes in the amount of fluid to be delivered. As operation of an electro-osmotic device is continued for example, the passage of molecules across the membrane of the cell causes a steady increase in the salt concentration within the first electrode cell and a steady decrease in the second electrode cell. The concentration difference typically allows environmental osmosis flux to develop. Ultimately, a steady-state delivery rate is reached due to establishment of steady-state concentrations in both half-cells. At steady-state, environmental osmosis becomes a significant component in the overall flux. The additional solvent transfer causes an increase in the overall fluid amount contained in the second half-cell containing the device product chamber, increasing the rate of fluid delivery.

One observed drawback of typical osmotic fluid delivery devices is that as the operation of a device is continued over a period of time, the delivery rate changes and becomes somewhat unreliable and inconsistent due to changes in the relative concentrations of salts within the osmotic compartment(s). Consequently, osmotic fluid delivery devices that address these changes in a manner that provides a greater level of control over the delivery rate of a fluid are desirable.

### SUMMARY OF THE INVENTION

The invention disclosed herein has a number of embodiments. Illustrative embodiments of the invention include an osmosis driven fluid delivery apparatus comprising at least one osmotic compartment that is typically coupled to a stationary semi-permeable membrane that permits fluid migration across the membrane and into the osmotic compartment. Certain embodiments of the invention have two or more osmotic compartments. In embodiments of the invention, the osmotic compartment(s) is adapted to include an initial chemical composition (e.g. one or more ion species) that functions to alter osmotic pressure within the osmotic compartment(s) upon fluid migration across the stationary semi-permeable membrane. The apparatus typically includes a displaceable barrier member coupled to the osmotic compartment(s), wherein the displaceable barrier member is displaced in response to alterations in osmotic pressure within the osmotic compartment(s); a medication reservoir including a fluid outlet for delivering a fluid medication from the medication reservoir, wherein the medication reservoir is coupled to the displaceable barrier member such that medication is delivered from the medication reservoir through the fluid outlet upon displacement of the displaceable barrier member. In certain embodiments of the invention, the osmosis driven fluid delivery apparatus comprises an electro-osmotic pump. In other embodiments of the invention, the osmosis driven fluid delivery apparatus does not comprise an electro-osmotic pump.

In order to modulate osmotic forces that drive fluid flow, embodiments of the invention include a solute reservoir including a fluid conduit that is capable of delivering a solute fluid from the solute reservoir into the one or more osmotic compartments, wherein delivery of the solute fluid into the osmotic compartment(s) functions to alter osmotic pressure within the osmotic compartment(s) in a manner that influences the delivery the fluid medication from the medication reservoir. Embodiments of the invention further include a solute delivery system that delivers the solute fluid from the solute reservoir into the osmotic compartment(s). Optionally such embodiments of the invention include a solute delivery controller that controls delivery of the solute fluid from the solute reservoir into the osmotic compartment(s). Certain embodiments of the invention also include one or more fluid bleed members that function to modulate the fluid volume in the osmotic compartment(s).

The solute reservoir used in embodiments of the invention provides both a new mechanism to precisely control osmotic pump function as well as new osmotic pump designs. For example, in the osmotic pump embodiment of the invention that is shown in FIG. 1, a steady-state mathematical model predicts that an implantable amplification device having such a solute reservoir can be constructed to convert a 1 uL/hr flowrate of saturated sodium chloride from the solute reservoir into a drug delivery rate of 28 uL/hour, (using 1 cm² of commercially available desalination membrane). Such embodiments of the invention can consequently provide for a 28-fold reduction in the size of the osmotic compartment(s), significantly reducing the overall volume required for the implanted apparatus. Other variations of this embodiment include the temporal manipulation of the infusion rate of solute into the osmotic compartment(s) to control the drug delivery rate. In certain embodiments of the invention, the fluid delivery apparatus is an electro-osmotic apparatus as shown for example in FIGS. 2 and 4.

Other embodiments of the invention include sealed electro-osmotic pump engines. One embodiment is an electro-osmotic pump design that does not discharge ions into the surroundings or require water from an external source. This embodiment of the invention addresses certain issues with previously disclosed electro-osmotic pump engines that utilize the host's body fluid as a water supply and further discharge potentially toxic ions. Conventional electro-osmotic drug delivery devices lack closed-device means to accommodate fluid transfer between anodic and cathodic osmotic compartments. Embodiments of the invention include an electrochemical cell that loses fluid an comprises an element comprising a piston or a flexible, bellows-like outer wall to accommodate fluid loss without exposing contents to extracellular space. Features of embodiments of the invention include a flexible anodic or cathodic half-cell wall (depending upon the pump type). In certain embodiments of the invention, this element is a moveable or deformable trap member. Optionally, this moveable or deformable trap member is coupled to the medication reservoir such that the capture materials such as ions produced in the function of the apparatus produce pressure within this member that resultantly drives fluid medication from the medication reservoir out of the fluid outlet.

Another embodiment of the invention is a method of modulating fluid delivery (e.g. fluid medication delivery) from a medication reservoir within a fluid delivery apparatus as disclosed herein. In this embodiment, the method comprises delivering an amount of a solute fluid from a solute reservoir into an osmotic compartment(s) of the apparatus, wherein the amount of fluid delivered from the solute reservoir into the osmotic compartment(s) is sufficient to alter the osmotic pressure within the osmotic compartment(s) so as to displace a displaceable barrier member and modulate delivery of a fluid medication from the medication reservoir through the fluid outlet. In an illustrative methodological embodiment of the invention, the amount of the solute fluid delivered from the solute reservoir into an osmotic compartment is sufficient to produce an oscillating fluid delivery profile. Optionally the solute fluid delivered from the solute reservoir into the osmotic compartment(s) is sufficient to produce a fluid medication delivery profile comprising a first amount of fluid medication delivered within hours 1-10 after initiating fluid delivery and a second amount of fluid medication delivered within hours 11-20 after initiating fluid delivery, wherein the first amount of fluid medication delivered within hours 1-10 is at least 2, 3, 4, 5, 7, 8 or 9 times the second amount of fluid medication delivered within hours 11-20. In some embodiments of the invention, fluid delivery (e.g. fluid medication delivery) is further controlled by the activation of a fluid bleed member that can modulate the fluid volume in at least one osmotic compartment of the apparatus. In a specific methodological embodiment of the invention, the stationary semi-permeable membrane in the apparatus is an ion selective membrane, an initial chemical composition in the an osmotic compartment(s) comprises the ion at a first concentration, and a solute fluid in the solute reservoir comprises the ion at a second concentration, and the first concentration and the second concentration are selected so that a first fluid flow rate from the solute reservoir into the osmotic compartment(s) produces a second fluid flow rate from the medication reservoir through the fluid outlet, wherein the second fluid flow rate is at least 2, 4, 6, 8, 10, 12, 14, 16, 18, 20, 22, 24, 26 or 28 times the first fluid flow rate.

Embodiments of the invention also provide articles of manufacture including pump elements, pump apparatus and kits. In one such embodiment of the invention, a kit including an osmotic pump apparatus or set, useful for delivering a fluid as is described above, is provided. The kit and/or pump apparatus typically comprises a container, a label and an osmotic pump apparatus as described above. The typical embodiment is a kit comprising a container and, within the container, an osmotic pump apparatus having a design as disclosed herein and instructions for using this osmotic or electro-osmotic pump apparatus.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and embodiments of the invention include all such modifications.

### BRIEF DESCRIPTION OF THE DRAWINGS

FIG. 1 provides an illustration of an embodiment of the invention that can be employed for the amplification of volumetric flow rates. This figure illustrates a fluid delivery apparatus 10 comprising: a first osmotic compartment 20 (Compartment 1) coupled to a stationary semi-permeable membrane 30. In this embodiment, a displaceable barrier member 40 is coupled to the first osmotic compartment and is displaced in response to alterations in osmotic pressure within the first osmotic compartment. This embodiment also includes a medication reservoir 50 including a fluid outlet 60 for delivering a fluid medication from the medication reservoir. This embodiment also includes a solute reservoir 70 including a fluid conduit 80 that is capable of delivering a solute fluid from the solute reservoir into the first osmotic compartment, wherein delivery of the solute fluid into the first osmotic compartment functions to alter osmotic pressure within the first osmotic compartment. In addition, this embodiment also includes a solute delivery system 90 that delivers the solute fluid from the solute reservoir into the first osmotic compartment; and a solute delivery controller 100 that controls delivery of the solute fluid from the solute reservoir into the first osmotic compartment. Typically, one or more of the components is disposed within a housing 300 of the apparatus.

In typical embodiments, a concentrated solution (or pure solute) can be delivered into at least one osmotic compartment or compartments to create, sustain, and/or modulate the osmotic pressure gradient across the stationary semi-permeable membrane. Options for the solute delivery system in such embodiments include, but are not limited to: traditional osmotic pumps such as the DUROS® device, electro-osmotic pumps such as Cation Electro-Kinetic devices, constant-flowrate devices, or other fluid delivery devices and systems known in the art (see e.g. U.S. Pat. Nos. 6,491,684, 6,575,961, 6,872,292 which are incorporated by reference herein). A steady-state mathematical model predicts that an implantable amplification device can be constructed that converts a 1 uL/hr flowrate of saturated sodium chloride into a drug delivery rate of 28 uL/hour (using 1 cm² of commercially available desalination membrane). It then follows that such embodiments can potentially result in a 28-fold reduction in the size of the osmotic compartment (or compartments), significantly reducing the overall volume of implanted fluid delivery systems. Another variation of this embodiment includes the temporal manipulation of the infusion rate of solute into an osmotic compartment(s) to control the drug delivery rate.

As is understood in the art and further noted below, the embodiments of the inventions described in the figures are merely illustrative and that embodiments of the invention can include a variety of combinations of elements that can be organized into a variety of functional configurations.

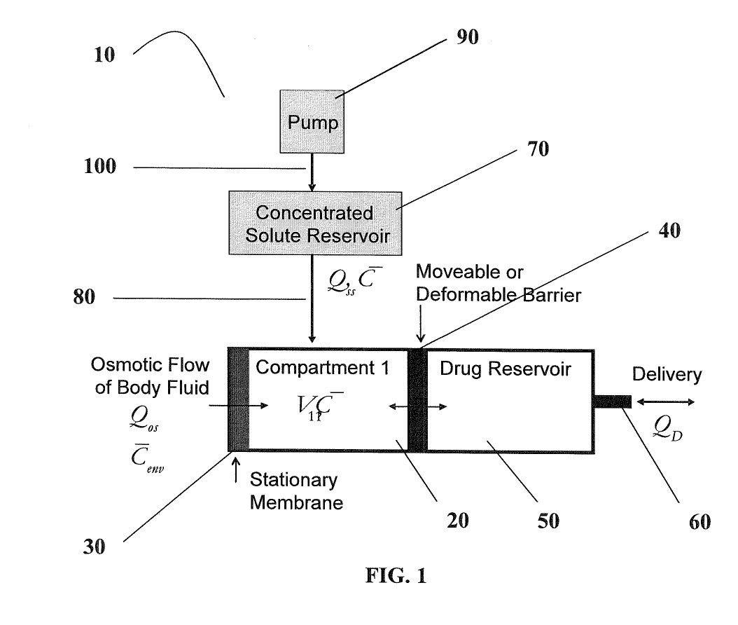

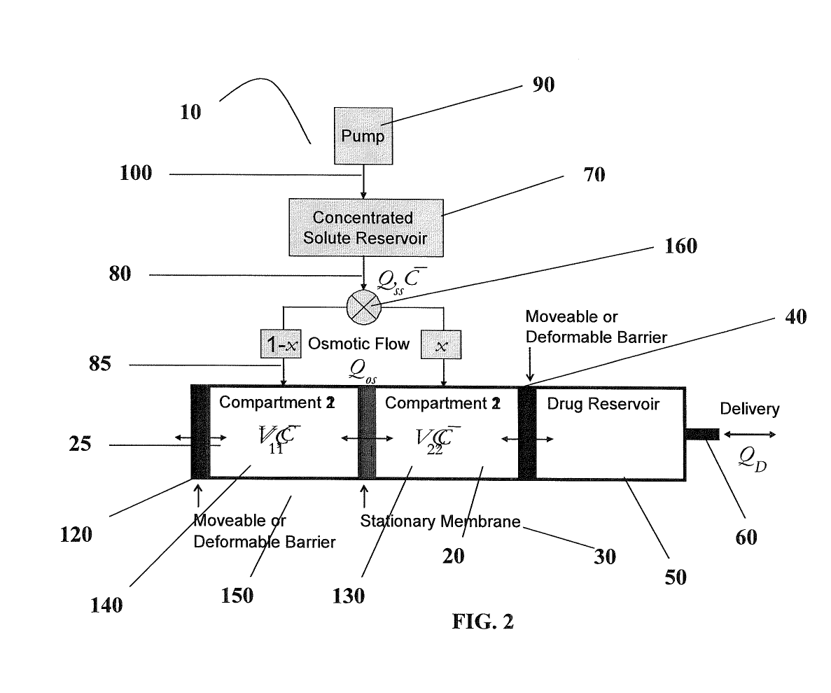

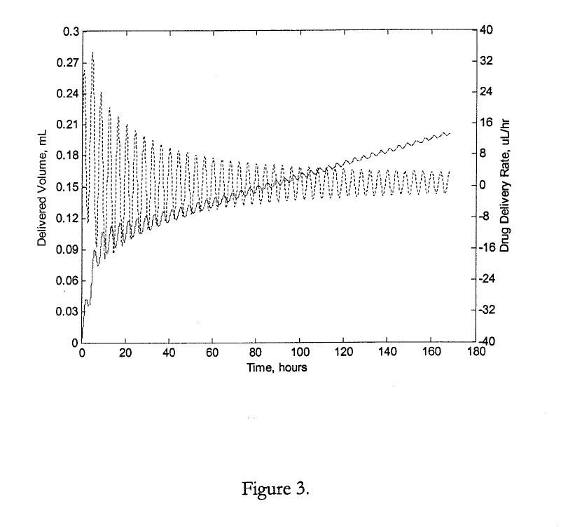

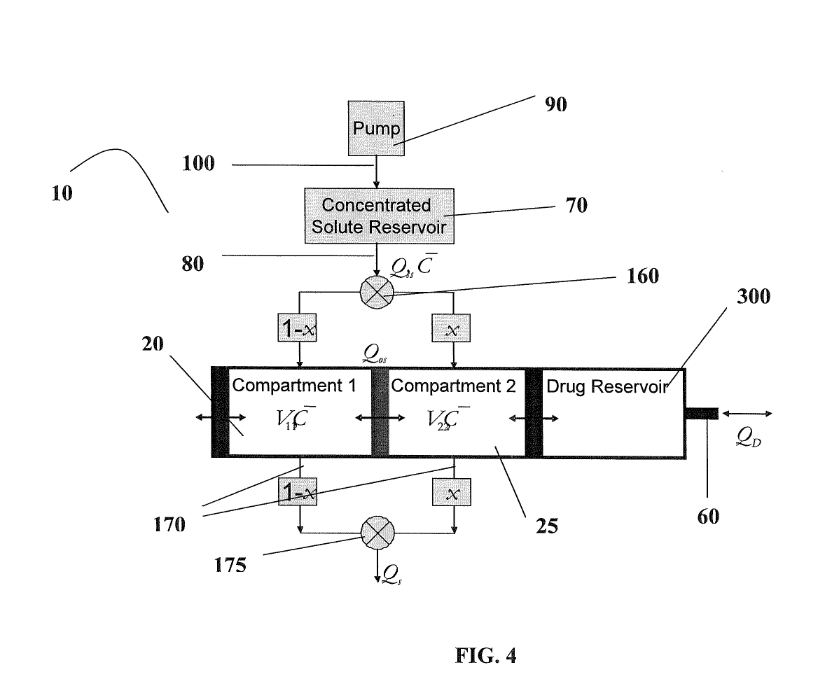

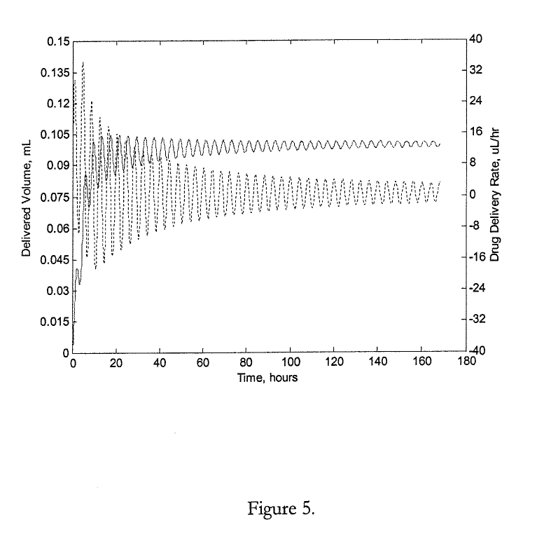

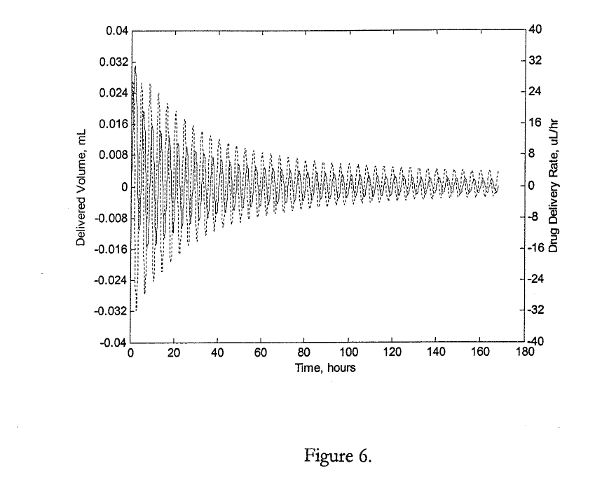

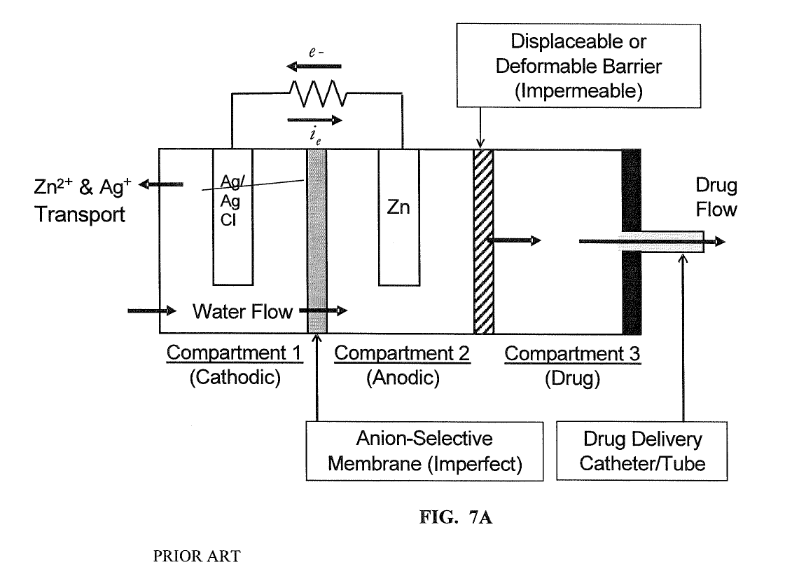

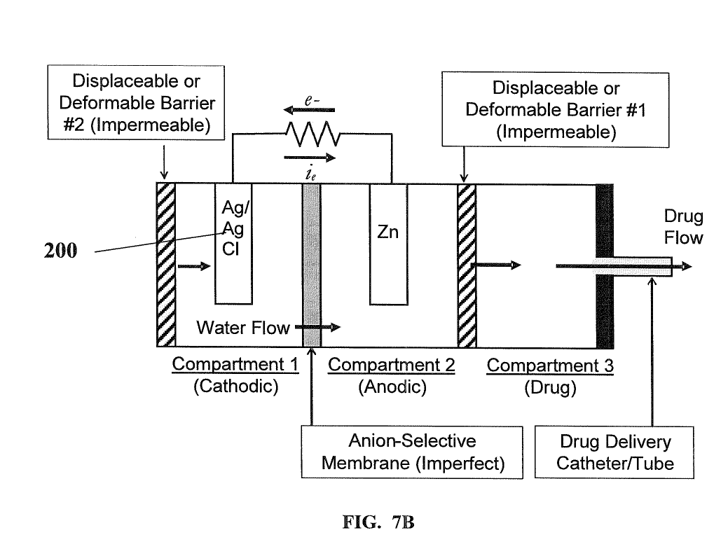

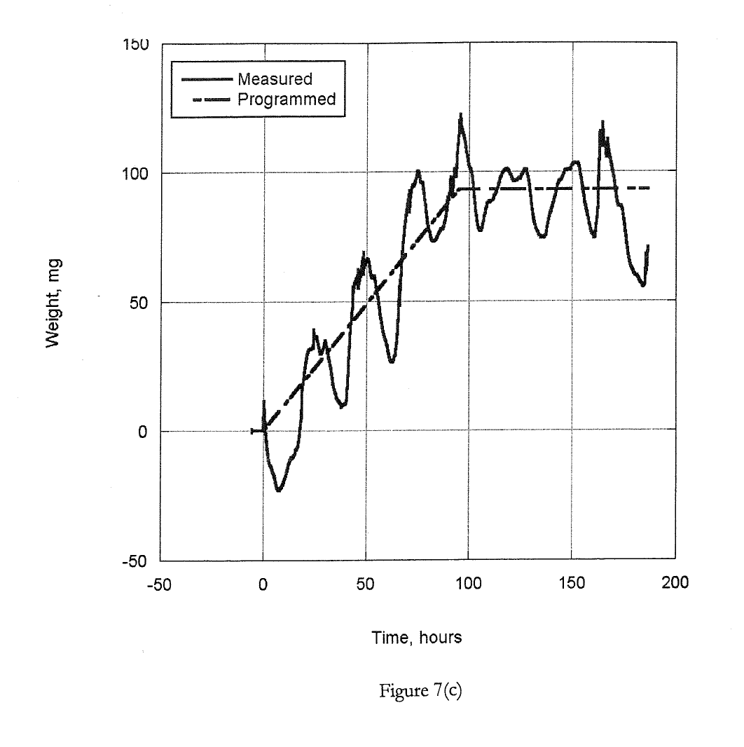

FIG. 2 provides an illustration of another embodiment of the invention that can be employed for the amplification, advanced control, and even reversal of volumetric flow rates. In addition to the elements shown in FIG. 1, this embodiment further includes a second osmotic compartment 25 (Compartment 2) coupled to a portion of the stationary semi-permeable membrane, wherein the second osmotic compartment contains a fluid capable of migrating from the second osmotic compartment across the stationary semi-permeable membrane into the first osmotic compartment. This embodiment can also include a second displaceable barrier member 120 coupled to the second osmotic compartment. In certain embodiments, the first osmotic compartment includes a first electrode 130 and the second osmotic compartment includes a second electrode 140 so as to form an electrochemical cell, wherein the first and second osmotic compartments include a fluid electrolyte in communication with the first and second electrodes and further wherein the first and second electrodes are coupled to a controller 150 that controls an electrical signal sent to or received from the first or second electrodes. The solute reservoir further includes a fluid conduit 85 capable of delivering a solute fluid from the solute reservoir into the second osmotic compartment, wherein delivery of the solute fluid into the second osmotic compartment modulates the osmotic pressure within the first osmotic compartment, the second osmotic compartment or the first osmotic compartment and the second osmotic compartment. The fluid conduit capable of delivering a solute fluid from the solute reservoir into the second osmotic compartment and/or the fluid conduit capable of delivering a solute fluid from the solute reservoir into the first osmotic compartment comprises a control element such as a valve 160 to direct or meter fluid flow in to these osmotic compartments.

In this embodiment, a concentrated solution (or pure solute) held in a solute reservoir can be delivered into osmotic Compartments 1 and/or 2. The flowrate split ratio (x) and total flowrate of concentrated solution (Qs) may be constant or time-dependent. For example, if Qs is held constant and x follows the temporal behavior described by Equation [1], the delivery profile is predicted by a mathematical model to be that illustrated by FIG. 3.

FIG. 3 provides a Model-Predicted Delivery Profile of a Reversible Osmotic Flow Modulator (FIG. 2). Model input parameters are as follows: xₘₐₓ=1.0, xₘₘ=0.2, V₁⁰=V₂⁰=0.5 mL, ω1.57 rad/hr, Φ=π/2 rad, Qₛ=1 μL/hr, C̅ₛ=10 M, T=310 K, Aₘ=1 cm², Pₘ=2.5×10⁻¹⁶ m³/Pa-s-cm², and C₁⁰=C₂⁰=10 mM.

In this model, the drug delivery system is predicted to deliver an initial net bolus of approximately 90 μL over the first 10 hours and then continue to deliver an average basal rate of 1 μL/hour. Furthermore, the instantaneous delivery rate of the system is predicted to follow an oscillatory pattern. This is useful when a dissolution chamber is used as the medication reservoir. The oscillatory instantaneous flowrate can be used to increase the ratio of the drug delivery rate (units/hour) to the net volumetric flowrate (μL/hour). This is useful for applications where it is desirable or necessary to minimize the net delivered volume. An example of such an application can be the delivery of baclofen, lidocane, or epidermal growth factor to the cochlea of the ear for the treatment of tinnitus or deafness.

FIG. 4 illustrates another embodiment of the invention that is a variation of that depicted in FIG. 2. In addition to the elements shown in FIG. 2, this embodiment further includes an arrangement of elements where the first osmotic compartment, the second osmotic compartment or the first osmotic compartment and the second osmotic compartment comprise a fluid bleed member 170 that can modulate the fluid volume in the first osmotic compartment, the second osmotic compartment or the first osmotic compartment and the second osmotic compartment. Therefore it differs from the embodiment shown in FIG. 2 in that it includes bleed flow streams that can be used to control the total volume of osmotic Compartments 1 and 2.

As illustrated by FIG. 5, this aspect of the design allows for the elimination of the basal delivery rate depicted in FIG. 3. The ability of this embodiment to eliminate the net delivered volume is demonstrated by FIG. 6, where the value of xₘᵢₙ has been changed from 0.2 to 0.0.

FIG. 5 provides a Model-Predicted Delivery Profile of an Osmotic Flow Modular with Bleed Flow (FIG. 4). Model input parameters are identical to FIG. 3: xₘₐₓ=1.0, xₘᵢₙ=0.2, V₁⁰=V₂⁰=0.5 mL, ω=1.57 rad/hr, φ=π/2 rad, Qₛ=1 μL/hr, C̅ₛ=100 M, T=310 K, Aₘ=1 cm², Pₘ=2.5×10¹⁶ m³/Pa-s-cm², and C₁⁰=C₂⁰=10 mM.

FIG. 6 provides a Model-Predicted Delivery Profile of an Osmotic Flow Modulator with Bleed Flow (FIG. 4) and Zero Net Volumetric Delivery. Model input parameters are identical to FIG. 5, except xₘᵢₙ=0.

FIGS. 7(a)-7(c) provides a schematic diagrams of: (a) Anion Electro-Kinetic type devices known in the art; (b) and a sealed Anion Electro-Kinetic type device including a deformable trap member 200 as disclosed herein; and (c) a graph showing fluid delivery into a prototype deformable trap member 200 comprising a balloon. This embodiment of a sealed electro-osmotic pump is similar to that shown in FIG. 2, and further includes a first 130 and second 140 electrode as well as a controller 150 that controls an electrical signal sent to or received from the first or second electrodes. FIG. 7(c) shows fluid delivery from a prototype embodiment of the invention comprising a 2 cc Microlin device with AFN membrane, 0.9% saline, room temperature and electrolyte balloon that includes moveable or deformable trap member 200 that captures ions or other components that would otherwise be released from the pump in to the external environment.

### DETAILED DESCRIPTION OF EMBODIMENTS OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted.

A number of terms are defined below.

“Fluid delivery device” and “Fluid delivery apparatus” as used herein refers to any device or apparatus suitable for delivering fluids to an individual such as fluidic therapeutic formulations. Such apparatuses and devices can for example be implantable, or alternatively, external (e.g. an external device carried by the user). These terms encompass any implantable device with any mechanism of action including diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems.

The term “subject” is meant any subject, generally a mammal (e.g., human, canine, feline, equine, bovine, etc.). The term “individual” is meant any single human subject.

“Treatment” or “therapy” refer to both therapeutic treatment and prophylactic or preventative measures.

The term “therapeutically effective amount” is meant an amount of a therapeutic agent, or a rate of delivery of a therapeutic agent, effective to facilitate a desired therapeutic effect. The precise desired therapeutic effect will vary according to the condition to be treated, the formulation to be administered, and a variety of other factors that are appreciated by those of ordinary skill in the art. In the case of infection, the therapeutically effective amount of the drug may reduce the number of infective agents (e.g. bacteria or viruses); inhibit to some extent, the growth of the infective agent; and/or relieve to some extent one or more of the symptoms associated with the infection. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. For cancer therapy, efficacy in vivo can, for example, be measured by assessing tumor burden or volume, the time to disease progression (TTP) and/or determining the response rates (RR).

The term “medication” as in “fluid medication” encompasses all medicinal agents suitable for delivery according to the methods of the invention, and is not meant to be limiting in any way. As used herein, this term broadly refers to any agent used to treat or facilitate the treatment, amelioration or diagnosis of a pathological condition. Illustrative fluid medications include polypeptide medications such as an interferon as well as antibodies such as anti-TNF-α antibodies that function to inhibit TNF-α activity. Fluid medications can comprise an antibiotic, antiviral or other growth inhibitory agent, a prodrug, a cytotoxic agent, a chemotherapeutic agent, a polypeptide such as a cytokine, combinations of these agents or the like. The term “fluid” is herein defined as a liquid, gel, paste, or other semi-solid state material that is capable of being delivered out of a reservoir (e.g. a medication, solute or water reservoir) of an osmotic pump apparatus.

A “growth inhibitory agent” when used herein refers to a compound or composition which inhibits growth of a cell or virus in vitro and/or in vivo. Such agents include antiviral, antibiotic and chemotherapeutic agents. Thus, a growth inhibitory agent may be one which kills or inhibits the growth of viruses or bacteria or one which significantly reduces the percentage of mammalian cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), TAXOL®, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C.

The term “prodrug” as used in this application refers to a precursor or derivative form of a pharmaceutically active substance that is less cytotoxic to cancer cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described below.

The term “cytotoxic agent” as used herein refers to a substance that inhibits or prevents the function of cells and/or causes destruction of cells. The term is intended to include radioactive isotopes (e.g., Ar²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

A “chemotherapeutic agent” is a chemical compound useful in the treatment of conditions like cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (₁¹ and calicheamicin 2¹₁, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhône-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

The term “cytokine” is a generic term for proteins released by one cell population which act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-alpha; platelet-growth factor; transforming growth factors (TGFs) such as TGF-alpha and TGF-beta; insulin-like growth factor-I and -II; erythropoietin (EPO); osteoinductive factors; interferons such as interferon-alpha, -beta and -gamma colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; a tumor necrosis factor such as TNF-alpha or TNF-beta; and other polypeptide factors including LIF and kit ligand (KT). As used herein, the term cytokine includes proteins from natural sources or from recombinant cell culture and biologically active equivalents of the native sequence cytokines.

The term “local delivery” is meant to encompass routes of delivery that result in a medication being delivered to a specific anatomical region. The term “systemic delivery” is meant to encompass all parenteral routes of delivery which permit a medication to enter into the systemic circulation, e.g., intravenous, intra-arterial, intramuscular, subcutaneous, intra-adipose tissue, intra-lymphatic, etc.

“Delivery site” as used herein is meant to refer to an area of the body to which drug is delivered, e.g., a site which allows local or systemic access of drug delivered to the site. Exemplary delivery sites compatible with local delivery include the cochlea of the inner ear. Exemplary delivery sites compatible with systemic delivery of drug include, but are not necessarily limited to, subcutaneous, intravenous, intra-arterial, intra-muscular, intra-adipose tissue, and intra-lymphatic sites. The term “implantation site” is used to refer to a site with the body of a subject at which a drug delivery device is introduced and positioned.

As discussed in detail below, the invention disclosed herein provides elements and combinations of elements that can be used with a wide variety of osmotic pump apparatuses and systems. In certain embodiments of the invention, the elements and combinations of elements disclosed herein are adapted for use with an electro-osmotic pump. Electro-osmotic pumps use an electrochemical cell and a membrane wherein during operation of the electrochemical cell there is a transport of water across the membrane to create a driving force for fluid flow to vary volume or pressure to displace a substance or fluid. In other embodiments of the invention, the elements and combinations of elements disclosed are adapted for use with a fluid delivery apparatus that is not an electro-osmotic pump. An osmotic fluid delivery apparatus is not an electro-osmotic pump when for example, it does not include an electrochemical cell to create a driving force for fluid flow to vary volume or pressure to displace a substance or fluid.

Embodiments of the invention are directed to apparatuses that utilize osmosis to function. Briefly, osmosis is the diffusion of a liquid (most often assumed to be water, but it can be any liquid solvent) through a semi-permeable membrane from a region of high chemical potential to a region of low chemical potential. The selectively-permeable membrane must be permeable to the solvent, but not to the solute, resulting in a pressure gradient across the membrane. The force per unit area required to prevent the passage of solvent through a selectively-permeable membrane and into a solution of greater concentration is equivalent to the turgor pressure. Osmosis can be controlled or modulated in a number of ways, e.g. by increasing the pressure in the section of high solute concentration with respect to that in the low solute concentration.

In operation, osmotic pumps imbibe water or other driving fluid. Such pumps typically consists of at least three chambers (e.g. reservoirs, compartments and the like): a salt chamber, a water chamber, and a fluid chamber. The salt and water chambers are separated by a semi-permeable membrane. This configuration creates a high osmotic driving force for water transport across the membrane. This membrane is permeable to water, but impermeable to salt. The fluid chamber is separated from the other two chambers by a flexible diaphragm. Water imbibes osmotically into the salt chamber creating substantial hydrostatic pressures, which in turn exert a force on the diaphragm—thus expelling the fluid.

Embodiments of the invention are directed to apparatuses that utilize osmosis to drive fluid delivery (e.g. delivery of a fluid medication). Typical osmotic and electro-osmotic pump engines known in the art are driven either entirely or in part by osmosis: the spontaneous transport of water from a dilute solution into a concentrated solution through a solute-impermeable membrane. The inner osmotic compartments of osmotic pumps (e.g. DUROS® device from Alza) are typically pre-loaded with a solution that is relatively concentrated as compared to the surrounding operational environment. Electro-osmotic pumps (e.g. a Cation Electro-Kinetic-type device from MicroLin) are believed to create concentrated solutions in their inner osmotic compartments via electrochemical processes.

Embodiments of the invention disclosed herein are directed to novel device designs for the creation and manipulation of the solute concentrations within the inner osmotic compartment (or compartments) of osmotic pumps. This manipulation of the solute concentrations within the inner osmotic compartment(s) of osmotic pumps can be used for example to precisely control fluid delivery from the pump. As shown below, the invention described herein has wide variety of embodiments. The illustrative embodiments disclosed in the text and drawings are not intended to limit the broad aspect of the invention to the embodiments illustrated. Instead, these illustrative embodiments are merely typical examples of embodiments of the invention.

As noted above, embodiments of the invention include fluid delivery apparatuses that utilize osmotic forces to deliver the fluid. Typically, the fluid delivery apparatus includes a solute reservoir that contains a composition designed to modulate osmotic forces in the apparatus, for example by altering the concentration of one or more ion species in one or more osmotic compartments within the apparatus that experiences the osmotic forces that deliver the fluid. In this context, the controlled delivery of a solute fluid from the solute reservoir into the one or more osmotic compartments within the apparatus that experiences osmotic forces that function to drive fluid delivery consequently modulates these osmotic forces, which resultantly modulates fluid delivery from the apparatus.

Surprisingly, the controlled delivery of a solute fluid from the solute reservoir into the one or more osmotic compartments within an osmotic pump apparatus exhibits an unexpectedly profound influence on pump performance. For example, as disclosed herein, a 1 uL/hr flowrate (Qs) of saturated sodium chloride (Cₛ=10 M) can be converted into a drug delivery rate (Q_{D}) of 28 uL/hour (using 1 cm² of commercially available desalination membrane). In this way, the instant disclosure (e.g. the mathematical modeling parameters) consequently allows artisans to consider and construct novel pump designs. One such embodiment of the invention enables a 28-fold reduction in the size of the osmotic compartment(s) of a sealed electro-osmotic pump engine. This significantly reduces the overall device volume, addressing a key limitation of sealed electro-osmotic pump designs. Other embodiments of the invention allow for the advanced control and reversal of volumetric flow rates. One embodiment is capable of delivering drugs with “zero net volumetric delivery” to volume-sensitive locations such as the cochlea of the inner ear. Other embodiments of the invention are able to deliver bolus, basal, reverse and periodic flow rates. Embodiments of the invention provide a number of advantageous properties, for example zero net volumetric delivery, a reduction of the medication reservoir volume required for a sealed electro-osmotic pump engine, as well as control over reverse, oscillatory or periodic flow.

Illustrative embodiments of the invention that can be employed for the amplification of volumetric flow rates are shown in FIG. 1 and FIG. 4. In such embodiments of the invention, a concentrated solution (or pure solute) is pumped into a first osmotic compartment (Compartment 1) and/or a second osmotic compartment (Compartment 2) to create, sustain, and/or modulate the osmotic pressure gradient across the stationary semi-permeable membrane. A wide variety of pumps known in the art can be adapted for use in such embodiments of the invention. Options for the pump in this embodiment include, but are not limited to: traditional osmotic pumps such as the DUROS® device, electro-osmotic pumps such as a Cation Electro-Kinetic device, or constant-flowrate devices known in the art. Further pump embodiments are discussed below.

In osmotic pump embodiments of the invention such as that shown in FIG. 1, a steady-state mathematical model indicates that an implantable amplification device can be constructed to greatly amplify flow rates without a coordinate amplification in the volume of a medication (e.g. a drug) reservoir. In one example, a 1 uL/hr flowrate (Qs) of saturated sodium chloride (Cₛ=10 M) converts into a drug delivery rate (Q_{D}) of 28 uL/hour (using 1 cm² of commercially available desalination membrane). This embodiment consequently provides for a 28-fold reduction in the size of the osmotic compartment(s), significantly reducing the overall volume of the implanted system. Another variation of this embodiment includes the temporal manipulation of the infusion rate (Cₛ×Qₛ) of solute into an osmotic compartment to control the drug delivery rate.

As discussed in detail below, embodiments of the invention that include a solute reservoir for modulating osmotic pressure include electro-osmotic pump apparatuses, for example those that include a first osmotic compartment and a second osmotic compartment that can function as a first half-cell and second half-cell, with an ion-selective membrane in-between. In such embodiments, a fluid inlet is associated with a first osmotic compartment and/or a second osmotic compartment, allowing fluid from the surrounding environment of fluid delivery device into the cell. Within first half-cell and second half-cell are electrodes. In order to regulate the operation of the electrochemical cell, the electrochemical cell typically includes means for controlling the electrochemical cell. In this context, FIG. 2 illustrates another electro-osmotic embodiment of the invention that can be employed for the amplification, advanced control, and even reversal of volumetric flow rates. In the embodiment shown in FIG. 2, a concentrated solution (or pure solute) is pumped into the first and/or second osmotic compartments (Compartments 1 and/or 2).

The flowrate split ratio (x) and total flowrate of concentrated solution (Qₛ) may be constant or time-dependent. For example, if Qₛ is held constant and x follows the temporal behavior described by Equation [1], the delivery profile is predicted by a mathematical model to be that illustrated by data shown in FIG. 3.

$$x ( t ) = \frac{{x}_{max} - {x}_{min}}{2} [ sin ( ω t + φ ) + 1 ] + {x}_{min} \qquad [ 1 ]$$

As shown by the mathematical modeling and associated data that is presented herein, embodiments of the invention can be used in a variety of methods designed to precisely control osmotic pump fluid delivery. For example, FIG. 3 illustrates a model of a drug delivery system and method designed to deliver an initial net bolus of approximately 90 μL over the first 10 hours and then continue to deliver an average basal rate of 1 μL/hour. Furthermore, the instantaneous delivery rate of the system is predicted to follow an oscillatory pattern. This is useful for example when a dissolution chamber is used as the medication (e.g. a drug) reservoir. The oscillatory instantaneous flowrate can be used to increase the ratio of the drug delivery rate (units/hour) to the net volumetric flowrate (μL/hour). This is useful for applications where it is desirable or necessary to minimize the net delivered volume. Example of such applications include the delivery of baclofen, lidocane, or epidermal growth factor to the cochlea of the ear for the treatment of tinnitus or deafness.

FIG. 4 illustrates another embodiment of the invention that is a variation of that depicted in FIG. 2. It differs in that it includes bleed flow streams that can be used to control the total volume of osmotic compartments 1 and/or 2. As illustrated by FIG. 5, this aspect of the design allows for the elimination of the basal delivery rate depicted in FIG. 3. The ability of this embodiment to eliminate the net delivered volume is demonstrated by FIG. 6, where the value of xₘᵢₙ has been changed from 0.2 to 0.0. Consequently, such elements can be used with embodiments of the invention to provide a further level of control over osmotic processes within osmotic pumps.

Other related embodiments of the invention can be readily constructed where the delivery of concentrated solution or pure solute to osmotic Compartment 1 of the embodiment shown in FIG. 1, or osmotic Compartment 2 and/or osmotic Compartment 2 in the embodiment shown in FIG. 2 is further affected by any number of existing controlled release technologies familiar to those skilled in the art, including, but not limited to: liposomes, polymeric matrices, ion-selective membranes, semi-permeable membranes, gas-generators, liquid-phase chemical reactions, heterogeneous chemical reactions, enzyme-substrate reactions, and delivery “microchip” technologies developed by MicroCHIPS (Bedford, Mass.). A variety of these embodiments are discussed below.

Typical embodiments of the invention include an osmosis driven fluid delivery apparatus comprising a first osmotic compartment coupled to a stationary semi-permeable membrane that permits fluid migration across the membrane and into the first osmotic compartment. In this embodiment, the first osmotic compartment is adapted to include an initial chemical composition (e.g. one or more ion species) that functions to alter osmotic pressure within the first osmotic compartment upon fluid migration across the stationary semi-permeable membrane. This is termed an “initial” chemical concentration because, as is known in the art, the concentration of the composition is not unchanged over time and instead changes during the osmotic process. This term is therefore used to precisely characterize the invention in accordance with mechanisms involved in the functioning of the apparatus. The apparatus also includes a displaceable barrier member coupled to the first osmotic compartment, wherein the displaceable barrier member is displaced in response to alterations in osmotic pressure within the first osmotic compartment; a medication reservoir including a fluid outlet for delivering a medication from the medication reservoir, wherein the medication reservoir is coupled to the displaceable barrier member such that fluid medication is delivered from the medication reservoir through the fluid outlet upon displacement of the displaceable barrier member. In order to modulate osmotic forces that drive fluid flow, the apparatus includes a solute reservoir including a fluid conduit that is capable of delivering a solute fluid from the solute reservoir into the first osmotic compartment, wherein delivery of the solute fluid into the first osmotic compartment functions to alter osmotic pressure within the first osmotic compartment. The apparatus further includes a pump that delivers the solute fluid from the solute reservoir into the first osmotic compartment; and a solute delivery controller that controls delivery of the solute fluid from the solute reservoir into the first osmotic compartment.

Other embodiments of the invention include sealed osmotic pump engines. One embodiment is an implantable osmotic pump design that does not discharge ions into the surroundings or require water from an external source. This embodiment of the invention addresses certain issues with previously disclosed osmotic pump engines that utilize the host's body fluid as a water supply and further discharge potentially toxic ions. Conventional osmotic drug delivery devices lack closed-device means to accommodate fluid transfer during osmosis. Embodiments of the invention include an osmotic compartment that loses fluid and comprises an element such as a piston or a flexible, bellows-like outer wall to accommodate fluid loss without exposing contents to extracellular space. In certain embodiments of the invention, this element is a moveable or deformable trap member. Optionally, this moveable or deformable trap member is coupled to the medication reservoir such that the capture materials such as ions produced in the function of the apparatus produce pressure within this member that resultantly drives fluid medication from the medication reservoir out of the fluid outlet.

A related embodiment is a sealed electro-osmotic pump engines. One embodiment is an electro-osmotic pump design that does not discharge ions into the surroundings or require water from an external source. This embodiment of the invention addresses certain issues with previously disclosed electro-osmotic pump engines that utilize the host's body fluid as a water supply and further discharge potentially toxic ions. Conventional electro-osmotic drug delivery devices lack closed-device means to accommodate fluid transfer between anodic and cathodic cells. Embodiments of the invention include an electrochemical cell that loses fluid and comprises an element such as a piston or a flexible, bellows-like outer wall to accommodate fluid loss without exposing contents to extracellular space. Features of embodiments of the invention include a flexible anodic or cathodic half-cell wall (depending upon the pump type). In certain embodiments of the invention, this element is a moveable or deformable trap member. Optionally, this moveable or deformable trap member is coupled to the medication reservoir such that the capture materials such as ions produced in the function of the apparatus produce pressure within this member that resultantly drives fluid medication from the medication reservoir out of the fluid outlet.

The sealed osmotic pump engine embodiments of the invention can be adapted for use with a wide variety of components used in osmosis based pump apparatuses. Additional components common to osmotic pumps include electrochemical half-cells separated by an ion-selective semi-permeable membrane. In an illustrative embodiment, the wall of the half-cell where fluid accumulates is coupled to a piston or a flexible, bellows-like wall acting against a medication reservoir, whereby fluid accumulating in the half-cell acts on the piston or a flexible, bellows-like wall to force fluid from the medication reservoir through a catheter and into the patient. Optionally such embodiments include an electrical energy source and control equipment to regulate pump flow, and can include sensors, programmers, or timers. Illustrative clinical applications include the localized delivery of biological TNF-α inhibitors for the treatment of sciatica and low back pain as well as the systemic delivery of interferon (e.g. Interferon alfa-2a, interferon alpha-2b and interferon alfacon-1) for the treatment of hepatitis C. These devices can deliver agents at either constant or variable specified rates.

FIG. 7(a) illustrates the basic design of an Anion Electro-Kinetic type electro-osmotic pump device. The design of Cation Electro-Kinetic type devices is similar, but employs a cation-selective membrane and transposed positions of the anode and cathode. Equations 2 and 3 describe the production of chloride anions and zinc cations at the cathode and anode, respectively.

2AgCl₍ₛ₎+2e^{-→}2Ag₍ₛ₎+2Cl_{(aq)}⁻ [2]

Zn₍ₛ₎→2e⁻+Zn_{(aq)}²⁺ [3]

In the Anion Electro-Kinetic type device, the first and second osmotic compartments are separated by an anion-selective membrane. During operation chloride anions and their solvating water molecules migrate from osmotic Compartment 1 into osmotic Compartment 2, with the net effect of producing zinc chloride in osmotic Compartment 2. The resulting osmotic pressure gradient drives the osmotic transport of water from osmotic Compartment 1 into osmotic Compartment 2, causing a medication such as a drug to be eluted from the medication reservoir. Because ion-selective membranes are imperfect, the zinc cations produced in osmotic Compartment 2 slowly diffuse through the anion-selective membrane into osmotic Compartment 1 and the surrounding environment. This mass transfer process is believed to affect shut-off of the pump engine.

FIG. 7(b) illustrates an illustrative sealed electro-osmotic pump engine embodiment of the invention. This embodiment is an Anion Electro-Kinetic type device, and is almost identical to that in FIG. 7(a), except that a movable or deformable member has been added that separates the contents of osmotic Compartment 1 from the surrounding environment, preventing the discharge of ions from the pump engine. In an exemplary embodiment, this member will consist of a low-friction integrated piston. In alternate embodiments it will consist of a diaphragm, bellows, or balloon. Other embodiments of the invention can consist of a Cation Electro-Kinetic-type device with a movable or deformable member that separates the contents of the anodic cell from the surrounding environment. FIG. 7(c), provides data from an apparatus prototype where the movable or deformable member is a balloon.

Optionally, embodiments of the osmotic pump apparatuses disclosed herein further include at least one one-way valve, also known as a check valve or anti-free-flow valve. In some embodiments of the invention, this check valve is used as an alternative to or in addition to the movable or deformable member described above as functioning to preventing the discharge of ions from the pump engine. Such valves can be used in embodiments of the invention to control the direction of fluid flow, for example the flow of fluid from an external environment such as a site of implantation into the osmotic pump apparatus. In addition, such check valves can be used in any embodiments of the invention where a conduit can be adapted to include a check valve to control the direction of fluid flow, for example the flow of fluids in to the osmotic apparatus, out of the osmotic apparatus, or between compartments within the osmotic apparatus. One such embodiment of the invention addresses certain issues with previously disclosed osmotic pump engines that utilize the host's body fluid as a water supply and further discharge potentially toxic ions. In this context, a check valve can be employed in an implantable apparatus so as to allow the apparatus to utilize the host's body fluid as a water supply (i.e. the movement of materials in one direction) but prevent the movement of materials in the opposite direction (e.g. the discharge potentially toxic ions into the host's body fluid).

Typical check valves described in the art are molded in a unitary fashion from a elastomeric composition such as silicone rubber. One illustrative embodiment is a circular valve disk having a protruding cylindrical dynamic sealing ridge on its top, which is the actual valve element. A static seal ring having a larger inner diameter than the outer diameter of the valve disc can be located concentrically around the valve disk. The valve disk is typically supported from the static seal ring by a support element such as a thin support web extending between the static support ring and the valve disk, which web has a plurality of holes that allow the passage of fluid. In operation, when the pressure is greater on top of the valve disk than under the valve disk, the valve will tend to open, requiring only a small pressure to operate. However, when this small break pressure is not present, or when a reverse pressure is present, the valve will remain in a closed position. The valve thus has a positive sealing action when closed, and opens easily when the small crack pressure (or a greater pressure in that direction) is present. Typically such valves are highly precise, for example operating in a passive manner to open with a relatively small break pressure or cracking pressure in the desired direction of flow through the valve. The valve is typically resistant to a substantially higher reverse pressure. A variety of check valves are well know in the art and described for example in U.S. Pat. Nos. 2,462,189, 2,497,906, 4,141,379, 4,593,720, 4,594,058, 4,657,536, 4,714,462, 4,846,787, 4,946,448, 5,527,307, 6,089,272 and 6,932,110, the contents of each of which are incorporated by reference.

Additional illustrative embodiments of the various elements of the invention are described in detail below. Artisans will understand that the apparatus and elements can be made from any of a wide variety of materials that are known in the art. For example, the osmotic compartment, reservoir(s) and housing elements can be fabricated from any one of a number of suitable materials, including metals, glass, natural and synthetic plastics as well as composites and the like.

Embodiments of the invention are useful as an implantable medical device for delivering a medicament to a patient over a period of time. Although the present invention is shown in conjunction with implantable devices, it should be noted that the teachings contained within the specification and the appended claims may be translated to other devices and applications without departing from the intended scope of this disclosure. In addition, embodiments of the invention can be adapted for use with a wide variety of fluid delivery apparatuses known in the art. While the elements are given common designations, analogous elements and/or components may be identified by comparing these elements to the elements shown in the drawings and reference characters. It is also to be understood that the embodiments shown in the FIGS. are merely a schematic representation of the osmotic delivery devices of the present invention.

The invention described herein has a wide variety of embodiments. A typical embodiment of a fluid delivery apparatus is shown in FIG. 1. This embodiment of the invention is a fluid delivery apparatus comprising: a first osmotic compartment coupled to a stationary semi-permeable membrane. In this embodiment, the stationary semi-permeable membrane permits fluid migration across the membrane and into the first osmotic compartment. The first osmotic compartment is adapted to include an initial chemical composition that functions to alter osmotic pressure within the first osmotic compartment upon fluid migration across the stationary semi-permeable membrane. One of skill in the art will understand that the term “initial” is used as in “initial chemical composition” because the composition changes over time, for example as part of the osmotic processes of the invention. This embodiment includes a displaceable barrier member coupled to the first osmotic compartment, wherein the displaceable barrier member is displaced in response to alterations in osmotic pressure within the first osmotic compartment; as well as a medication reservoir including a fluid outlet for delivering a fluid medication from the medication reservoir, wherein the medication reservoir is coupled to the displaceable barrier member such that fluid medication is delivered from the medication reservoir through the fluid outlet upon displacement of the displaceable barrier member. Optionally the fluid outlet comprises a fluid conduit such as a catheter that directs the fluid (e.g. the fluid medication) to a specific site, for example one that is distal (or alternatively proximal) to the in vivo site where the apparatus is implanted. Embodiments of the invention further includes a solute reservoir including a fluid conduit that is capable of delivering a solute fluid from the solute reservoir into the first osmotic compartment, wherein delivery of the solute fluid into the first osmotic compartment functions to alter osmotic pressure within the first osmotic compartment; a pump that delivers the solute fluid from the solute reservoir into the first osmotic compartment; and a solute delivery controller that controls delivery of the solute fluid from the solute reservoir into the first osmotic compartment. Typically, the apparatus further includes a housing. Optionally the housing is coated with one or more agents to promote biocompatibility, for example a heparin composition, a steroid such as dexamethasone or a polypeptide such as hirudin. It will be further understood that FIG. 1 is merely a schematic representation of fluid delivery apparatus. As such, some of the components have been distorted from their actual scale for pictorial clarity.

Certain embodiments of the invention comprise electro-osmotic pumps and include elements associated with their function. In an illustrative embodiment, the fluid delivery apparatus comprises a second osmotic compartment coupled to a portion of the stationary semi-permeable membrane, wherein the second osmotic compartment contains a fluid capable of migrating from the second osmotic compartment across the stationary semi-permeable membrane into the first osmotic compartment. In this embodiment, the first and second osmotic compartments function as an electro-osmotic cell, with the two osmotic compartments functioning as a first and second half-cell. Within the first half-cell and the second half-cell are electrodes with a first electrode in the first half-cell and second electrode in the second half-cell. This electro-osmotic cell includes an electrolyte in electrical communication with both the first electrode and the second electrode, enabling operation of the cell. In order to regulate the operation of the electrochemical cell, the apparatus typically includes an electrical controller for controlling the electrochemical cell.

Embodiments of the invention include one or more stationary semi-permeable membranes. The stationary semi-permeable membranes can be used to allow the passage of fluids between the external environment (e.g. body fluids of an individual) and the apparatus or between osmotic compartments within the apparatus. The membrane generally comprises an ion-selective or ion-exchange membrane that allows the passage of the ions, while substantially maintaining the integrity between an osmotic compartment(s) and fluids in the external environment. The particular material selected for membrane will depend on the exact configuration and function of the apparatus. For example, for electro-osmotic pumps, the particular material selected for membrane is typically dictated by the electrode materials selected and the desired pumping rate of fluid delivery device. Typical materials for such membranes include perfluorosulfonate membranes known in the art and available under the trade name NAFION. Additional resins are the copolymers of styrene and di-vinyl benzene having sulphonate ion as the charge group which has high selectivity sodium ions. Exemplary materials further include Neosepta type membranes, C/R, CMB, CMB-2, C66-F, and CCG-F, AM-1, AM-3 AFN and AM-X from Ameridia CM-1, CM-2, CMB, and others, commercially available from AMERIDIA, CMI 7000, Membranes International and PC-200D from PCA GmBH.

In one electro-osmotic pump embodiment of the invention, an anion exchange membrane is positioned between the first electrode and the second electrode. The anion exchange materials from which the membrane may be made are well known in the art and include cross-linked polymer resins of the strong base type. Typical resins are the copolymers of styrene and di-vinyl benzene having quaternary ammonium ion as the charge group, which have a high selectivity for chloride ions and high resistance to organic fouling. Such anionic membranes are, for example, Neosepta-type membranes, which are commercially available from AMERIDIA. Alternatively, a cation exchange membrane is used. The cation exchange materials from which the membrane may be constructed are well known in the art and include cross-linked polymer resins of the strong base type. Some typical resins include copolymers of styrene and di-vinyl benzene having sulfonate ion as the charge group, which have a high selectivity for sodium ions. Such commercial cationic membranes, e.g., Nafion type membranes, are available from Dupont.

In certain embodiments of the invention, the stationary semi-permeable membrane is exposed to a body fluid of the individual and the apparatus uses water in the body fluid of the individual to modulate osmotic pressure within the apparatus as the water migrates across the stationary semi-permeable membrane into the first osmotic compartment. In some embodiments of the invention, the apparatus comprises a water reservoir that is coupled to the stationary semi-permeable membrane, wherein the water modulates osmotic pressure within the apparatus as the water migrates across the stationary semi-permeable membrane into the first osmotic compartment. Optionally a portion of the stationary semi-permeable membrane is disposed on the apparatus to be exposed to fluid in an external environment, such that a fluid in the external environment can migrate across the stationary semi-permeable membrane into the first osmotic compartment. In certain embodiments of the invention, at least one osmotic compartment within the osmotic pump is preloaded with solutions having discreet ion combinations and/or concentrations that are selected to facilitate pump function.

Embodiments of the invention can include a protective porous separator that can for example function to inhibit clogging or fouling of apparatus components such as the stationary semi-permeable membrane. In one illustrative embodiment of the present invention, an anionic exchange membrane, the first electrode, the anionic exchange membrane, and the second electrode are respectively positioned adjacent to the protective porous separator. An alternate second embodiment of the present invention incorporates a cationic exchange membrane, with the first electrode, the cationic exchange membrane, and the second electrode are respectively positioned adjacent to the protective porous separator. Optionally, the protective porous separator further modulates water uptake by one or more processes such as convection or capillary action.

Generally, osmotic delivery device is associated with a water-rich environment (e.g. an in vivo environment) so that water may be allowed into the cell, optionally through the protective porous separator. In such embodiments of the invention, a protective porous separator can be positioned at an end of an apparatus housing a first half-cell and distally from an ion-exchange membrane. Thus, the protective porous separator is at least permeable to H₂O and NaCl molecules, and enables water and ions from an external source e.g., an inside of a living being's body, to migrate into the first half-cell. The protective porous separator may be fabricated from any of a number of materials, including, but not limited to: metals, glass, porous protective gel, natural and synthetic plastics, and composites. The use of the separator is not required and, accordingly, when not used, the first electrode can be exposed directly to fluid, if desired.

In alternative embodiments, the first electrode need not be positioned inside the device and can be positioned either entirely away from the housing or on the outside wall of the device. In such embodiments, the ion exchange membrane has more direct access to the body fluid and a porous separator can be placed directly adjacent to the ion-exchange membrane to prevent biofouling and to prevent unwanted species from contacting the membrane directly. This configuration will also eliminate trapping of any unwanted solid, liquid, or gaseous species in the auxiliary chamber and near the membrane. While the use of the protective porous separator may be generally desirable for applications within the body, the separator is not required, especially in the case where necessary water or saline is self-contained in an electrode cell without any migration of water from external source. In such embodiments, the first half-cell retracts or collapses around the electrode on transfer of water from the first half-cell to second half-cell via electro-osmosis. In such an embodiment, the first half-cell can be exposed directly to fluid.

Embodiments of the invention include a displaceable barrier member positioned to be coupled with the first osmotic compartment (and/or second osmotic compartment) and the medication reservoir. A variety of elements for use as displaceable barrier member are known in the art. Typically, the displaceable barrier member is a piston, a bellows, a bladder, a diaphragm, a plunger or a balloon or combinations thereof. In the fluid delivery apparatus, the displaceable barrier member is coupled to a medication reservoir having at least one outlet, exit aperture or port. During operation, the displaceable member is moveably associated within the device so that, as the volume of fluid contained within the first osmotic compartment increases, the displaceable member is correspondingly maneuvered into the medication reservoir, resulting in the reservoir's expulsion of fluid medication through the fluid conduit and into the external environment (e.g. a site of implantation within an individual). In an illustrative embodiment, the displaceable member is a piston which is positioned between the first osmotic compartment and the medication reservoir. In this context, the fluid medication reservoir is capable of containing a fluid medication, such as a drug or drug combination which is/are delivered via operation of the osmotic delivery apparatus. The term “fluid” broadly refers to any liquid, gel, paste, or other semi-solid state material that is capable of being delivered out of a fluid reservoir (e.g. a solute, medication or water reservoir) and outside of, or alternatively into portions of the apparatus.

Embodiments of the invention include a solute reservoir adapted for use in an osmotic pump apparatus. Typically, the solute reservoir is adapted for use in an osmotic pump apparatus by including a composition containing one or more compounds that function to modulate the osmotic pressure in one or more compartments of an osmotic pump apparatus. The solute reservoir is typically adapted for use in an osmotic pump apparatus by including a fluid conduit that is capable of delivering a solute fluid from the solute reservoir into an osmotic compartment, wherein delivery of the solute fluid into the first osmotic compartment functions to alter osmotic pressure within the first osmotic compartment. The solute reservoir can also be adapted for use in an osmotic pump apparatus by including a solute delivery controller that controls delivery of the solute fluid from the solute reservoir into the an osmotic compartment of an osmotic pump apparatus.

An illustrative embodiment of the invention is a solute reservoir having a fluid conduit that is capable of delivering a solute fluid from the solute reservoir into at least one osmotic compartment of the apparatus (e.g. the first or second osmotic compartments), wherein delivery of the solute fluid into the osmotic compartment functions to alter osmotic pressure within an osmotic compartment so as to ultimately effect fluid delivery from the apparatus. Contemplated embodiments of the invention include those having multiple solute reservoirs containing multiple compositions for modulating osmotic pressure. A wide variety of solute fluids can be used in such embodiments and such fluids typically contain a composition that alters the concentrations of at least one ion within an osmotic compartment of the apparatus. One example of such a solute fluid is a highly concentrated form of an ion composition used by an osmotic mechanism of the pump. Embodiments of the invention further include a solute delivery system that delivers the solute from the solute reservoir into the first osmotic compartment; and a solute delivery controller that controls delivery of the solute fluid from the solute reservoir into the first osmotic compartment. As is known in the art, such solute delivery systems can include fluid pumps as well as other fluid delivery systems known in the art.

One illustrative solute delivery system for use with embodiments of the invention includes a chamber or a plurality of chambers that are filled with a swelling agent that expands upon contact with water. The swelling agent is initially stored inside a chamber or chambers that is hermetically sealed and which can be opened individually on-demand. In this embodiment of the invention, certain aspects of the solute delivery system are similar to systems used in drug delivery technologies known in the art (see, e.g. U.S. Pat. Nos. 5,999,848, 6,551,838, 6,491,666, 6,527,762, U.S. Patent Application No. 20040106914 and Santini, et al. Nature 397, 28 Jan. 1999, the contents of each of which are incorporated by reference). Briefly, in this drug delivery technology, a substrate is constructed which contains a large number of chambers, each containing a drug. A barrier such as a gold foil membrane covers each chamber to produce a sealed chamber. When an aliquot of drug is desired, an electrical pulse can be delivered to one or more of the foil membrane(s) which results in the drug eluting out of the chamber.

In certain embodiments of the invention, these solute delivery systems function by including a chamber or a plurality of chambers that are filled with a swelling agent that expands upon contact with water. A barrier covers each chamber containing the swelling agent to produce a sealed chamber. When solute delivery is desired, the sealed chamber is opened which then results in the swelling agent eluting out of the chamber and into a space within the system that contains water. This swelling that results from the swelling agent's contact with water then produces a force which drives the solute fluid from the solute reservoir into an osmotic compartment of the apparatus.

A wide variety of swelling agents can be used in such embodiments of the invention. The swelling agent typically consists of one or more swellable hydrophilic polymers. Suitable swellable hydrophilic polymers include cellulose derivatives such as hydroxy C₁₋₄ alkyl celluloses, hydroxy C₁₋₄ alkyl C₁₋₄ alkyl celluloses, carboxyalkyl celluloses and the like; vinyl pyrrolidone polymers such as crosslinked polyvinylpyrrolidone or crospovidone; copolymers of vinyl pyrrolidone and vinyl acetate; gums of plant animal, mineral or synthetic origin such as agar, alginates, carrageenan, furcellaran derived from marine plants, guar gum, gum arabic, gum tragacanth, karaya gum, locust bean gum, pectin derived from terrestrial plants, microbial polysaccharides such as dextran, gellan gum, rhamsan gum, welan gum, xanthan gum, and synthetic or semi-synthetic gums such as propylene glycol alginate, hydroxypropyl guar and modified starches like sodium starch glycolate. The swellable hydrophilic polymers are present in suitable amounts such that the polymeric swelling agent exhibits controlled swelling and the desired rate of drug delivery is obtained and the polymeric swelling agent does not contribute significantly to increasing the size of the osmotic system. The polymeric swelling agent can comprise one or more of the above swellable hydrophilic polymers. Often, a mixture of two hydrophilic polymers provides the desired controlled swelling. Illustrative cellulose derivatives that may be used as swellable hydrophilic polymers in the polymeric swelling agent of the present invention include hydroxy C₁₋₄ alkyl celluloses such as hydroxymethyl cellulose, hydroxyethyl cellulose, hydroxypropyl cellulose and the like. For example, the polymeric swelling agent may be a mixture of two different types or two different grades of the hydroxy C₁₋₄ alkyl celluloses. In another embodiment of the present invention, copolymers of vinyl pyrrolidone and vinyl acetate, in admixture with alkylene oxide homopolymers such as polypropylene oxide, preferably ethylene oxide homopolymers or in admixture with hydroxy C₁₋₄ alkyl celluloses, preferably hydroxyethyl cellulose, may be used as the polymeric swelling agent. A wide variety of polyethylene polymers (e.g. polyethylene glycols) are commercially available.

A wide variety of pumps known in the art can be adapted for use in delivering a solute fluid from the solute reservoir into an osmotic compartment of the apparatus. Typical pumps include conventional mechanical and related pump designs known in the art. There are a number of implantable drug delivery pumps and systems presently being used that can be adapted for use with the instant invention and an illustrative (but not limiting) description of illustrative pumps that may be utilized with the invention is provided below.

One pump widely used in implantation is the programmable electromechanical SynchroMed® pump. Smaller sized implantable drug delivery pumps such as the osmotic pump of the DUROS® system may also be adapted for use with embodiments of the invention. In the operation of this pump, water is imbibed osmotically through a membrane into a salt chamber pressurizing a piston to expand into a drug chamber to force a drug out through a delivery orifice. The driving force behind the drug delivery of this pump is osmotic pressure, which can be as high as 200 atmospheres depending on the salt used, even though the pressure required to pump the drug from the device is small and the drug delivery rate remains constant as long as some excess undissolved salt remains in the salt chamber. In comparison with mechanically driven devices, osmotic systems are small, simple, reliable, and less expensive to manufacture. Because of the small size of the osmotic system, it can be implanted during a simple procedure in the physician's office.

Gas generating devices known in the art that are both portable and accurate for dispensing small volumes can be adapted to transport a fluid such as a solute fluid within embodiments of the invention. These gas-generating methods include galvanic cells and electrolytic cells. In galvanic gas generating cells, hydrogen or oxygen gas is formed at the cathode or anode, respectively, as a result of a reaction between a metal or metal oxide and an aqueous electrolyte. By definition, a galvanic cell is an electrochemical cell that requires no externally applied voltage to drive the electrochemical reactions. Typically, the anode and cathode of the galvanic cell are connected through a resistor that regulates the current passed through the cell, and in turn, directly regulates the production of gas that exerts a force on a diaphragm or piston—thereby expelling the drug. A number patents have disclosed delivery systems based on the use of galvanic hydrogen generating cell, e.g., U.S. Pat. Nos. 5,951,538; 5,707,499; and 5,785,688, the contents of each of which are herein incorporated by reference. In the cells disclosed in these patents, a zinc anode reacts with an alkaline electrolyte producing zinc oxide and water molecules are reduced on porous carbon electrode producing gaseous hydrogen. Additionally, U.S. Pat. Nos. 5,242,565 and 5,925,030 (the contents of each of which are herein incorporated by reference) disclose a galvanic oxygen-generating cell that is constructed much like a zinc/air button cell, wherein a reducible oxide is reduced at the cathode while hydroxyl ions are formed. The hydroxyl ions oxidize at the anode and release oxygen.

In contrast to galvanic cells, an electrolytic cell requires an external DC power source to drive the electrochemical reactions. When voltage is applied to the electrodes, the electrolyte gives off a gas that exerts a force on a diaphragm or piston-thus expelling the fluid. A number of electrolytic gas generating cells have been proposed for use in fluid delivery devices. A first type is based on water electrolysis requiring an operating voltage over 1.23 V. A second type, also known as oxygen and hydrogen gas pumps, requires a lower DC voltage than that utilized in water electrolysis systems. Both of these cell types utilize an ion exchange polymer membrane. A third type of gas generating electrolytic cell is based on the use of an electrolytically decomposable chemical compound that produces a reduced metal at the cathode, and generates gaseous oxygen by oxidation of water at the anode.

U.S. Pat. No. 5,891,097 (the contents of which are herein incorporated by reference) discloses an electrochemically driven fluid dispenser based on the electrolysis of water. Devices of this type can also be adapted to transport a fluid such as a solute fluid within embodiments of the invention. In this dispenser, water is contained in an electrochemical cell in which porous metal electrodes are joined to both sides of a solid polymer cation exchange membrane, and both of the two electrodes are made to contact with the water so as to use oxygen or hydrogen generated from an anode or cathode respectively, upon current conduction. Thus, hydrogen, oxygen, or a gas mixture of hydrogen and oxygen—generated by electrolysis of water when a DC current is made to flow between the electrodes—is used as a pressurization source of the fluid dispenser. Electrochemical oxygen and hydrogen pumps are constructed in a similar manner to the above-discussed water electrolysis cell and are described in several U.S. patents, e.g., U.S. Pat. Nos. 5,938,640; 4,902,278; 4,886,514; and, 4,522,698, the contents of each of which are herein incorporated by reference. Electrochemically driven fluid dispensers disclosed within these patents have an electrochemical cell in which porous gas diffusion electrodes are joined respectively to the opposite surfaces of an ion exchange membrane containing water functioning as an electrolyte. The electrochemically driven fluid dispenser uses such a phenomenon that when hydrogen is supplied to an anode of the electrochemical cell and a DC current is made to flow between the anode and the cathode, the hydrogen becomes hydrogen ions at the anode. When the produced hydrogen ions reach the cathode through the ion exchange membrane, an electrochemical reaction arises to generate gaseous hydrogen thereat. Since the net effect of these processes is the transport of hydrogen from one side of the membrane to the other, this cell is also called a hydrogen pump. The hydrogen generated and pressurized at the cathode is used as a driving source for pushing a piston, a diaphragm, or the like.

Embodiments of the invention utilize osmotic forces to function and can employ multiple osmotic pump mechanisms in a single apparatus, for example a first osmotic pump mechanism that is adapted to drive a solute from a solute reservoir into a first or second osmotic compartment of a second osmotic pump mechanism, with the second osmotic pump mechanism adapted to drive delivery of a fluid (e.g. a fluid medication) in to the external environment (e.g. a site of implantation). Alternatively, the pump that is adapted to drive a solute from a solute reservoir into a first or second osmotic compartment is not an osmotic pump.

Embodiments of the invention include a solute delivery controller that controls delivery of the solute fluid from the solute reservoir into the first osmotic compartment and/or the second osmotic compartment. In one embodiment of the invention, the solute delivery controller is a mechanism that actuates or modulates the function of a solute fluid pump such as a control switch. Alternatively, the solute delivery controller can comprise one of the variety of other fluid control elements known in the art such as a valve. In this way, the solute delivery controller 100 controls the delivery of the solute fluid from the solute reservoir in to the first osmotic compartment and/or the second osmotic compartment in a manner that consequently alters the osmotic forces in the first osmotic compartment and/or the second osmotic compartment in a manner that modulates fluid delivery (e.g. a fluid medication) from the device into the external environment.

As noted above, embodiments of the apparatus rely upon osmosis to drive or deliver a fluid such as a fluid medication from the inside of the pump into an external environment. As noted above, osmotic forces are altered during passage of the anions or cations through the semi-permeable membrane, where water is entrained with the ions so that an additional amount of water is transported into an osmotic compartment such as the first osmotic compartment. As the ionic membrane is an exchange for a specific type of ions, only those ions (e.g. cations) can pass through membrane. Therefore, water may be transported through the membrane only in one direction from, for example, an external environment (e.g. body fluids) or an internal reservoir such as a water reservoir or a second osmotic compartment to a first osmotic compartment that is coupled to a displaceable barrier member.

In certain embodiments, the fluid delivery apparatus further comprises a second osmotic compartment coupled to a portion of the stationary semi-permeable membrane, wherein the second osmotic compartment contains a fluid capable of migrating from the second osmotic compartment across the stationary semi-permeable membrane into the first osmotic compartment. Embodiments of the invention include a variety of permutations using such stationary semi-permeable membranes, for example an embodiment wherein the first osmotic compartment contains a fluid capable of migrating from the first osmotic compartment across the stationary semi-permeable membrane into the second osmotic compartment. Optionally the apparatus includes a second displaceable barrier member coupled to the second osmotic compartment. In electro-osmotic pump embodiments of the invention, the first osmotic compartment includes a first electrode and the second osmotic compartment includes a second electrode so as to form an electrochemical cell, and the first and second osmotic compartments include a fluid electrolyte in communication with the first and second electrodes. Typically, the first and second electrodes are coupled to a controller that controls an electrical signal sent to or received from the first or second electrodes.

An illustrative electro-osmotic cell of the invention comprises a first half-cell and second half-cell, with ion-selective membrane in-between. A fluid inlet is associated with first half-cell, allowing fluid from the surrounding environment of fluid delivery device into the cell. Within first half-cell and second half-cell are electrodes, with a first electrode in the first half-cell, and a second electrode in the second half-cell. The first electrode and second electrode typically comprise an anode and a cathode electrode. Alternatively, the first electrode can comprise a cathode, and second electrode can comprise an anode, depending upon the materials selected for the electrodes and membrane, and the operation of the fluid delivery device. Thus, these electrodes are interchangeable within first half-cell and second half-cell of the cell, depending upon the particular materials used for first electrode and the second electrode and for the semi permeable membrane. Some embodiments of the invention include additional electrodes known in the art so be utilized with the various devices and components disclosed herein.

One embodiment of the invention is an electro-osmotic cell having an improved mechanism for the cessation of cell operations after removal of operational current. The electro-osmotic cell includes a cell housing with a first half cell and a second half cell, which are separated by an ion-exchange membrane. Within each half cell is an electrode; a first electrode within the first half cell, and a second electrode within the second half cell. The electro-osmotic cell also includes an electrolyte in electrical communication with the first electrode and the second electrode, and a wiring apparatus electrically connecting the first electrode and the second electrode. All of these elements ensure the normal operation of the electro-osmotic cell. Additionally, however, the electro-osmotic cell includes means for counteracting at least some of the effects of salt concentration increases within the electro-osmotic cell associated with the wiring apparatus.

Such an electro-osmotic cell can beneficially be utilized within an electro-osmotic fluid delivery device. The above-described cell, along with all of the typical embodiments of that cell, can deliver fluid by combining the cell with a fluid inlet, a movable barrier such as a piston member adjacent the electro-osmotic cell, and a medication reservoir adjacent the piston member/movable barrier, the medication reservoir comprising a exit port. Typically the fluid inlet comprises a membrane (such as a permeable membrane or osmotic membrane), or a fluid conduit. Also, the piston member/movable barrier optionally comprises a slideable piston, a flexible diaphragm or the like. Typically, an electrolyte used with an osmotic cell can include a solution containing Na⁺ and/or K⁺ and Cl⁻ ions, such as fluid from a body (where the solvent is water and the electrolytes are naturally-occurring salt ions such as sodium and chloride ions) that can be delivered from the surrounding tissues to an implanted fluid delivery device. Alternatively, a number of other electrochemically compatible fluids can similarly be used (e.g., Ringer's solution, renal dialysis solution, PBS etc).

In a specific embodiment of the electro-osmotic apparatus of the present invention, the first electrode is comprised of porous silver chloride, manganese dioxide, or other materials that can be readily reduced or may catalyze a reduction reaction, e.g., reduction of oxygen or evolution of gaseous hydrogen from water-when coupled with the active metal anode. The second electrode is comprised of an active metal anode that can be a solid pellet, mesh, or metal powder type electrode fabricated from, for example, zinc, iron, magnesium, aluminum, or another corrosion stable metal or alloy. The ion-exchange membrane separating the first and second electrodes is an anion exchange membrane. The anionic exchange materials from which the membrane may be made are well known in the art and do not require extensive elaboration. Exemplary materials include polymeric membranes with styrene-divinyl benzene backbone with quaternary ammonia charge groups. Embodiments of the invention further include a solute reservoir 70 having a fluid conduit 80 that is capable of delivering a solute fluid from the solute reservoir into an electrode containing compartment of the apparatus (e.g. the first or second osmotic compartments), wherein delivery of the solute fluid into at least one osmotic compartment functions to alter osmotic pressure within an osmotic compartment so as to ultimately effect fluid delivery from the apparatus.

In some embodiments of the invention, in order to optimize operation of the cell, and to ensure that the occurrence of osmotic transfer (non-electro-osmotic) both during and after operation is minimized, both the anode and the cathode may be constructed from the same active materials. For example, in one embodiment, both the cathode and anode can comprise an Ag/AgCl electrode. In an illustrative embodiment the cathode produces a chloride ion, which is then passed across the membrane to the anode half-cell, whereafter the anode recomplexes the chloride ion into insoluble silver chloride, which then precipitates out of solution. In doing so, the concentration of the salt, namely the chloride ion, does not increase during operation, as it is complexed out of solution continuously. In addition, water is also transported with the chloride ions when current is flowing, resulting in a net volume flux into second half cell, and therefore fluid delivery from medication reservoir. Although the above embodiment solely describes the use of silver/silver-chloride active material electrodes, any other number of active materials can similarly be available for use as electrodes. As would be understood by one of ordinary skill in the art, simple experimentation can produce numerous other active materials for use in the present invention—provided the electrodes operate to help maintain a substantially constant salt concentration within the cell during operation.

A wide variety of electrode combinations can be utilized in various embodiments of the invention. In one embodiment, the first electrode is an anode, the second electrode is a cathode, and the membrane is cationic selective membrane. Alternatively, the first electrode can be a cathode, the second electrode an anode, and the membrane is anionic selective membrane. Anode materials may be of any suitable material to which a cation will migrate in a given electrolytic reaction, and may include materials such as carbon, platinum, zinc, magnesium, manganese, aluminum, silver, and silver/silver chloride. Cathode materials can include carbon, platinum, zinc, magnesium, manganese, aluminum, silver, and silver/silver chloride, among others. As with the dual-electrode embodiment, a single first electrode and a single second electrode optionally include a sensing means for detecting ionic concentration within the cell. Numerous materials can be used for both first electrode and second electrode, but they must be electrochemically compatible with one another so as to allow for the flow of ions and electrons during cell operation. Typical electrode material pairings can include, among others, Zn/Ag/Agl, Pt/Pt, Ag/AgCl/Pt, Zn/Pt, Pt/Ag/AgCl, Ag/AgCl/Ag/AgCl, and Zn/AgCl. In one embodiment, first electrode comprises a zinc electrode, and second electrode comprises an Ag/AgCl electrode.

The electrochemical cells used in embodiments of the invention typically include a controller for controlling the electrochemical cell. The controller can comprise a resistor, a control circuit, or the like. These devices help to control the time course and magnitude of current that flows through the electrodes of the electrochemical cell. In one embodiment, the electrochemical cell includes two or more second electrodes, wherein at least one of the two second electrodes optionally comprise substantially the same active material as the first electrode. Typically, the controller directs the flow of electricity between the first electrode and at least one of the two or more second electrodes. The flow of current may be directed either by splitting the current between the two second electrodes, or cycling the flow of current between the electrodes, as may be needed. In order to facilitate the simultaneous operation of the at least two second electrodes, the wiring loops for each electrode can include one or more resistors. The electrochemical cell may additionally include an ionic sensor for measuring the ionic concentration of at least one of the two half cells. This concentration can then be used to determine the operation of the controlling means.

In an illustrative embodiment of the invention, the controller is connected to the first electrode and the second electrode and comprises an electrical circuit, e.g., an activation switch, a control circuitry, and a resistor. The controller facilitates control of the time course and magnitude of current that flows through the electrodes of the electro-osmotic cell. The controller is also capable of adjusting the delivery rate in various manners and wave forms. Additionally, the controller can aid in fast shutoff of fluid delivery as described in U.S. Patent Application Publication No. US2004/0144646; the contents of which are incorporated herein by reference. Typically the electrical controller facilitates control of the rate of delivery of fluid out of the medication reservoir. In certain embodiments, the electrical controller, in cooperation with the activation switch, control circuitry, and resistor, are operably coupled to the first electrode and second electrode via conventional electrical conduit to control the rate of water transfer from the external source to the second half-cell, as well as the starting, stopping, and length of the operation. It is to be understood that the resistor may be substituted or augmented with other elements known in the art. The controller can be powered by power source so that, once a switch is closed (the operation of which may be controlled by the controller), operation of the apparatus is commenced. Alternatively, the controller can comprise power source itself.

In certain embodiments of the invention, the controller can additionally comprise sensor situated in the wall of an anodic or cathodic half-cell such that it is in direct contact with the solution contained therein. The sensor can be capable of detecting the conductivity of the fluid in half cell or the concentration of any number of ionic species contained within a half-cell, but especially should be able to detect and measure the ionic concentration of the ion produced by an anode or cathode during operation. Typical sensors include conductivity sensors, sodium ion sensors, Ag/AgCl chloride ion sensor, etc.

As shown in FIG. 4, embodiments of the invention can include one or more bleed flow streams (e.g. a fluid bleed member) that can be used to control the total volume in the first and second osmotic compartments. Such fluid bleed elements of the invention can be coupled to any compartment within an osmotic apparatus to direct fluid out of an to modulate pressure within that compartment. In addition, the fluid bleed elements can be used to direct fluid from any compartment within the osmotic apparatus to any other compartment within the osmotic apparatus, or alternatively to direct fluid outside of the osmotic apparatus. Optionally the fluid bleed member comprises a fluid conduit such as a catheter that directs a fluid to a specific site, for example one that is distal (or alternatively proximal) to the in vivo site where the apparatus is implanted. Optionally, the fluid conduit directs the fluid into a moveable or deformable trap member. Such bleed flow streams can be controlled by a wide variety of elements known in the art such as valves. The bleed valves can be controlled by timers, as well as pressure and/or chemical (e.g. ion) sensors. In an illustrative embodiment, a miniature solenoid valve bleeds fluid from first and/or second osmotic compartments to effect an ionic and/or pressure differential between the osmotic compartments, and in this way modulates the pressure on the medication reservoir. The valve(s) can be under the control of an electronic module, which includes for example a transducer signal processing and valve and pump driving electronics. The system can be powered for example from a DC supply through leads, either an external source connected to terminals, or a battery.

In certain embodiments of electro-osmotic pump apparatuses, due to the continuous formation of ions such as sodium chloride and zinc chloride, the steady buildup of ion concentration internally induces further water transport through environmental osmosis. Thus, a steady state flux of water transport is established over a period of time by the combined osmotic and electro-osmotic effects. The osmotic flux is the result of the necessary concentration gradient and can be modified by virtue of modifying the electro-osmotic driving force.

The following discussion of a specific embodiment of an electro-osmotic pump apparatus and the processes involved in its function illustrates the advantages of embodiments of the instant invention, for example a solute reservoir containing a concentrated ion composition and a mechanism for introducing this composition into at least one osmotic compartment of the apparatus. In operation, fluid delivery apparatus can deliver a fluid such as a fluid medication in accordance with the following process. Initially, an activation switch of the electrical controller is actuated, whereupon an electrical circuit is complete which causes electrode reactions to take place at the first and second electrodes, and water to be extracted from external environment, and, ultimately to be driven across ion exchange membrane into an osmotic compartment in the apparatus. Thus, water from external environment, such as a human body diffuses into an electrode containing compartment. In this way such devices and processes enable a controlled delivery of a fluid over an extended period of time at a relatively precise and accurate rate inasmuch as the water transported is proportional to the current, which in turn depends on a number of factors including the properties of the electrical controller (e.g. the value of a resistor of the electrical controller). Therefore, the fluid delivery rate can be controlled by selection of elements such as resistor and not only by the rate at which water is permitted to enter the housing of the apparatus.

Although such electro-osmotic delivery apparatuses that are described in the art are effective in delivering fluid through electro-osmotic transport, the amount of time required to achieve a consistent fluid delivery rate can be quite long. During operation, an increase in the salt concentration within one of the half-cells, e.g., second half-cell, can be observed, which can adversely affect electro-osmotic cell operations by causing additional osmotic transport within the cell. The slow buildup of steady-state ion concentration translates into slow establishment of steady state flux at the start of the operation of the device. This additional transport slowly increases until steady-state concentrations are reached in both the half-cells. A variety of methods can be utilized to control pump function and for example achieve an enhanced delivery profile such as a faster delivery startup. One method involves the electro-osmotic cell having a pre-configured concentration gradient so that one of the half-cells contains a higher concentrated solution than the other. Another method achieves a faster delivery startup by utilizing a controller to pass higher current between the two half-cells at the onset of the device operation.

As disclosed herein, yet another method for further controlling the delivery profile of osmotic apparatuses is by utilizing an apparatus having a constellation of elements that includes a solute reservoir including a fluid conduit that is capable of delivering a solute fluid from the solute reservoir into the first osmotic compartment, wherein delivery of the solute fluid into the at least one osmotic compartment functions to alter osmotic pressure within the first and/or second osmotic compartments. In such embodiments of the invention, a pump delivers the solute fluid from the solute reservoir into the osmotic compartment(s). Typically such embodiments of the invention include a solute delivery controller 100 that controls delivery of the solute fluid from the solute reservoir into the osmotic compartment(s). In osmotic pump embodiments of the invention such as that shown in FIG. 1, a steady-state mathematical model predicts that an implantable amplification device can be constructed that converts a 1 uL/hr flowrate of saturated sodium chloride into a drug delivery rate of 28 uL/hour, (using 1 cm² of commercially available desalination membrane). Such embodiments of the invention provides for a 28-fold reduction in the size of the osmotic compartment(s), significantly reducing the overall volume of the implanted system. Other variations of this embodiment includes the temporal manipulation of the infusion rate of solute into the osmotic compartment(s) to control the drug delivery rate.

Artisans understand that a variety of permutations and/or modifications can be made to the apparatuses disclosed herein. A typical embodiment is a fluid delivery apparatus (e.g. an implantable apparatus) comprising a first osmotic compartment coupled to a stationary semi-permeable membrane (e.g. an ion selective membrane), wherein the stationary semi-permeable membrane permits fluid migration across the membrane and into the first osmotic compartment. In this embodiment, the first osmotic compartment is adapted to include an initial chemical composition (e.g. an ion solution) that functions to alter osmotic pressure within the first osmotic compartment upon fluid migration across the stationary semi-permeable membrane. A displaceable barrier member 40 is coupled to the first osmotic compartment and is displaced in response to alterations in osmotic pressure within the first osmotic compartment. A medication reservoir including a fluid outlet for delivering a fluid medication from the medication reservoir is coupled to the displaceable barrier member such that fluid medication is delivered from the medication reservoir through the fluid outlet upon displacement of the displaceable barrier member. Optionally, the medication reservoir contains a medication selected from the group consisting of a drug, a lubricant, a surfactant, a disinfectant or mixtures thereof.

This embodiment includes a solute reservoir including a fluid conduit that is capable of delivering a solute fluid from the solute reservoir into the first osmotic compartment, wherein delivery of the solute fluid into the first osmotic compartment functions to alter osmotic pressure within the first osmotic compartment as well as a pump that delivers the solute fluid from the solute reservoir into the first osmotic compartment; and a solute delivery controller that controls delivery of the solute fluid from the solute reservoir into the first osmotic compartment. Optionally the stationary semi-permeable membrane is exposed to a body fluid of the individual and the apparatus uses water in a body fluid of the individual to modulate osmotic pressure within the apparatus as the water migrates across the stationary semi-permeable membrane into the first osmotic compartment. In some embodiments, the apparatus further comprises a water reservoir that is coupled to the stationary semi-permeable membrane, wherein the water modulates osmotic pressure within the apparatus as the water migrates across the stationary semi-permeable membrane into the first osmotic compartment. In some embodiments of the invention, a portion of the stationary semi-permeable membrane is disposed on the apparatus to be exposed to a fluid in an external environment, such that the fluid in the external environment can migrate across the stationary semi-permeable membrane into the first osmotic compartment.

Embodiments of the invention include electro-osmotic pumps and can include a second osmotic compartment coupled to a portion of the stationary semi-permeable membrane, wherein the second osmotic compartment contains a fluid capable of migrating from the second osmotic compartment across the stationary semi-permeable membrane into the first osmotic compartment. Optionally, a second displaceable barrier member coupled to the second osmotic compartment. Typically in such embodiments, the first osmotic compartment includes a first electrode and the second osmotic compartment includes a second electrode so as to form an electrochemical cell, and the first and second osmotic compartments include a fluid electrolyte in communication with the first and second electrodes. The first and second electrodes are coupled to a controller that controls an electrical signal sent to or received from the first or second electrodes. In such embodiments, the fluid conduit capable of delivering a solute fluid from the solute reservoir into the first and/or second osmotic compartments, wherein delivery of the solute fluid into the osmotic compartment modulates the osmotic pressure within the first osmotic compartment, the second osmotic compartment or the first osmotic compartment and the second osmotic compartment. In certain embodiments of the invention, the fluid conduit capable of delivering a solute fluid from the solute reservoir into the second osmotic compartment and/or the fluid conduit capable of delivering a solute fluid from the solute reservoir into the first osmotic compartment comprises a valve to direct or meter fluid flow. In embodiments of the invention, the first osmotic compartment and the second osmotic compartment comprise a chemical reagent which expands upon a chemical and/or electrochemical reaction. In one illustrative embodiment of the invention, the first and second electrodes comprise an anode and a cathode--and vice versa--and are separated by an ionic-exchange membrane placed there between. Typically, the ion-exchange membrane is situated within the housing and between the two (half-cell) compartments. Alternatively, the first half-cell need not be positioned inside the device and can be positioned either on the outside wall of the device or entirely away from the housing. In such a configuration, the first half-cell is directly exposed to the body fluid and a porous separator can be placed directly adjacent to the ion-exchange membrane.

Optionally, the first osmotic compartment, the second osmotic compartment or the first osmotic compartment and the second osmotic compartment comprise a fluid bleed member that can modulate the fluid volume in the first osmotic compartment, the second osmotic compartment or the first osmotic compartment and the second osmotic compartment. In certain embodiments, a fluid bleed member comprises a valve to direct or meter fluid flow. In certain embodiments of the invention, the operation of the apparatus produces ions that are released into a moveable or deformable trap member (e.g. a piston, a bellows, a bladder, a diaphragm, a plunger or a balloon or combinations thereof) so that the ions are not released into the body of the individual (i.e. when implanted). Optionally the moveable or deformable trap member is coupled to the medication reservoir such that fluid medication is delivered from the medication reservoir through the fluid outlet upon displacement of the moveable or deformable trap member.

In some embodiments of the invention, the solute delivery controller that controls delivery of the solute fluid from the solute reservoir into the first osmotic compartment or the second osmotic compartment is programmable and includes one or more solute delivery schedules, for example a solute delivery schedule that produces an oscillatory fluid delivery profile. Optionally, the apparatus further comprises a fluid medication disposed in the medication reservoir and the solute delivery controller that controls delivery of the solute fluid from the solute reservoir into the first osmotic compartment or the second osmotic compartment is programmable and includes a solute delivery schedule that produces a bolus of a fluid medication within 24 hours of initiation. In a specific embodiment of the invention, the stationary semi-permeable membrane is an ion selective membrane, the chemical composition in the first osmotic compartment comprises the ion at a first concentration, and a solute fluid in the solute reservoir comprises the ion at a second concentration, and the first concentration and the second concentration are selected so that a first fluid flow from the solute reservoir into the first osmotic compartment produces a second fluid flow from the medication reservoir through the fluid outlet, wherein the second fluid flow is at least 2, 4, 6, 8, 10, 12, 14, 16, 18, 20, 22, 24, 26 or 28 times the first fluid flow.

Another illustrative embodiment of the invention is a fluid delivery apparatus comprising a first osmotic compartment having a first electrode and a second osmotic compartment having a second electrode, wherein the first and second osmotic compartments are coupled to a stationary semi-permeable membrane, wherein the first and second osmotic compartments include a fluid electrolyte in communication with the first and second electrodes and further wherein the first and second electrodes are coupled to a controller that controls an electrical signal sent to or received from the first or second electrodes; and wherein the first osmotic compartment is adapted to include an initial chemical composition that functions to alter osmotic pressure within the first osmotic compartment or second osmotic compartments upon fluid migration across the stationary semi-permeable membrane. This embodiment includes a displaceable barrier member coupled to the first osmotic compartment, wherein the displaceable barrier member is displaced in response to alterations in osmotic pressure within the first or second osmotic compartments. The embodiment also includes a medication reservoir including a fluid outlet for delivering a fluid medication from the medication reservoir, wherein the medication reservoir is coupled to the displaceable barrier member such that fluid medication is delivered from the medication reservoir through the fluid outlet upon displacement of the displaceable barrier member. This embodiment also includes a moveable or deformable trap member adapted to capture ions produced in the function of the apparatus so that the ions are not released into the body of the individual. Optionally this embodiment includes a solute reservoir and associated control elements. In some embodiments, the apparatus further comprises a water reservoir that is coupled to the stationary semi-permeable membrane, wherein the water modulates osmotic pressure within the apparatus as the water migrates across the stationary semi-permeable membrane into the first osmotic compartment. In one embodiment, the moveable or deformable trap member is coupled to the medication reservoir such that captured ions produced in the function of the apparatus produces pressure that drives fluid medication out of the fluid outlet. Optionally the first osmotic compartment, the second osmotic compartment or the first osmotic compartment and the second osmotic compartment comprise a fluid bleed member that can modulate the fluid volume in the first osmotic compartment, the second osmotic compartment or the first osmotic compartment and the second osmotic compartment. Optionally, the fluid bleed member directs fluid into the moveable or deformable trap member.

Yet another embodiment of the invention is a method of modulating fluid delivery from a medication reservoir within a apparatus as disclosed herein. In this embodiment, the method comprises delivering an amount of a solute fluid from a solute reservoir into an osmotic compartment of the apparatus, wherein the amount of fluid delivered from the solute reservoir into the osmotic compartment is sufficient to alter the osmotic pressure within the osmotic compartment so as to displace a displaceable barrier member and modulate delivery of the fluid medication from the medication reservoir through the fluid outlet. In an illustrative embodiment of the invention, the amount of the solute fluid delivered from the solute reservoir into the first or second osmotic compartments is sufficient to produce an oscillating fluid delivery profile. Optionally the solute fluid delivered from the solute reservoir into the osmotic compartment(s) is sufficient to produce a fluid medication delivery profile comprising a first amount of fluid medication delivered within hours 1-10 after initiating fluid delivery and a second amount of fluid medication delivered within hours 11-20 after initiating fluid delivery, wherein the first amount of fluid medication delivered within hours 1-10 is at least 2, 3, 4, 5, 7, 8 or 9 times the second amount of fluid medication delivered within hours 11-20. In some embodiments of the invention, fluid delivery (e.g. fluid mediation delivery) is further controlled by the activation of a fluid bleed member that can modulate the fluid volume in an osmotic compartment of the apparatus.

In a specific methodological embodiment of the invention, the stationary semi-permeable membrane in the apparatus is an ion selective membrane, a chemical composition in the first osmotic compartment comprises the ion at a first concentration, and a solute fluid in the solute reservoir comprises the ion at a second concentration, and the first concentration and the second concentration are selected so that a first fluid flow rate from the solute reservoir into the first osmotic compartment produces a second fluid flow (e.g. fluid flow rate or fluid amount) from the medication reservoir through the fluid outlet, wherein the second fluid flow rate is at least 2, 4, 6, 8, 10, 12, 14, 16, 18, 20, 22, 24, 26 or 28 times the first fluid flow (e.g. fluid flow rate or total volume of fluid).

The apparatus of the invention can be configured according to its intended use, for example for implantation at a specific in vivo location. The housing can take a variety of forms, for example an elongated cylindrical containing the first half-cell and the second half-cell. The housing may be constructed of metal, glass, natural and synthetic plastics, composites, or a combination thereof. Optionally, the first half-cell is positioned between the ion-exchange membrane and the protective porous separator or protective gel, and is capable of containing water and electrolytic products that are controllably generated during the initiation of the current. The second half-cell can be positioned between a displaceable member and the first half-cell, and be capable of containing water and electrolytic products that are controllably generated during operation of first half-cell. One or more support member(s) can be configured proximate the ion-exchange membrane and the first half-cell. The support member(s) can provide mechanical rigidity for components such as the ion-exchange membrane and allow water to transport through it. The support member can be made of hard plastic, ceramic, glass, corrosion stable metal (e.g., titanium), or other like materials known to those with ordinary skilled in the art.

While specific embodiments of the present invention have been illustrated and described, numerous modifications come to mind without significantly departing from the spirit of the invention and the scope of protection is only limited by the scope of the accompanying claims. All publications listed in the specification are hereby incorporated by reference. Embodiments of the invention can be adapted for use with a variety of the different types of osmosis systems (e.g. those utilizing various ion systems) known in the art. Elements, methods and materials of such systems are disclosed for example in U.S. Pat. No. 3,760,984, U.S. Pat. No. 3,971,376, U.S. Pat. No. 3,987,790, U.S. Pat. No. 3,995,631, U.S. Pat. No. 3,995,632, U.S. Pat. No. 4,410,328, U.S. Pat. No. 6,568,910, U.S. Pat. No. 6,572,749, U.S. Pat. No. 6,575,961, U.S. Pat. No. 6,491,684, U.S. Pat. No. 6,872,292, U.S. Pat. No. 6,689,373, U.S. Pat. No. U.S. Pat. No. U.S. Pat. No. U.S. Pat. No. U.S. Pat. No.; U.S. Pat. No. 3,894,538, U.S. Pat. No. 3,893,904, U.S. Pat. No. 4,140,121, U.S. Pat. No. 4,140,122, U.S. Pat. No. 4,687,423, U.S. Pat. No. 5,163,899, U.S. Pat. No. 6,004,309, U.S. Pat. No. 6,206,659, U.S. Pat. No. 5,585,069, U.S. Pat. No. 5,593,838, U.S. Pat. No. 5,454,922, U.S. Pat. No. 5,603,351, U.S. Pat. No. 5,632,876, U.S. Pat. No. 5,643,738, U.S. Pat. No. 5,681,484, U.S. Pat. No. 5,755,942, U.S. Pat. No. 5,858,804, U.S. Pat. No. 5,863,708, U.S. Pat. No. 5,980,704, U.S. Pat. No. 6,331,439, U.S. Pat. No. 6,159,171, U.S. Pat. No. 6,313,164, U.S. Pat. No. 5,924,848, U.S. Pat. No. 5,938,412, U.S. Pat. No. 6,012,902, U.S. Pat. No. 6,171,067, U.S. Pat. No 6,394,759, U.S. Pat. No. 6,568,910, U.S. Pat. No. 5,454,922, U.S. Pat. No. 5,567,287, U.S. Pat. No. 5,538605, U.S. Pat. No. 5,427,870, U.S. Pat. No. 5,593,522, U.S. Pat. No. 5,855,761, U.S. Pat. No. 5,997,821, U.S. Pat. No. 5,707,499, U.S. Pat. No. 6,042,704, U.S. Pat. No. 5,785,688, U.S. Pat. No. 5,744,014, U.S. Pat. No. 5,932,204, U.S. Pat. No. 6,060,196, U.S. Pat. No. 5,951,538, U.S. Pat. No. 6,109,539, U.S. Pat. No. 6,045,055, U.S. Pat. No. 6,283,461, U.S. Pat. No. 6,220,267, U.S. Pat. No. 6,327,426 U.S. Pat. No. 6,591,133, U.S. Pat. No. 6,787,008, U.S. Pat. No. 7,047,069, U.S. Pat. No. 6,575,961, U.S. Pat. Application No. 2003023187, U.S. Pat. Application No. 2003028124, U.S. Pat. Application No. 2003040682, U.S. Pat. No. 6,059,736, U.S. Pat. No. 6,485,437, U.S. Pat. No. 6,843,254, U.S. Pat. No. 6,019,882, U.S. Pat. No. 6,277,257, U.S. Pat. No. 6,572,749, U.S. Pat. Application No. 2003171401, U.S. Pat. Application No. 2004157884, U.S. Pat. Application No. 2005106205, U.S. Pat. Application No. 2005129737, U.S. Pat. No. 6,541,021, U.S. Pat. No. 6,689,373, U.S. Pat. No. 6,613,211, U.S. Pat. Application No. 2003140976, U.S. Pat. Application No. 2004094220, U.S. Pat. No. 6,675,821, U.S. Pat. Application No. 2002070116, U.S. Pat. Application No. 20020156461, U.S. Pat. No. 6,575,961 , U.S. Pat. Application No. 2002175191, U.S. Pat. No. 6,491,684, U.S. Pat. Application No. 2004208751, U.S. Pat. No. 6,460,974, U.S. Pat. Application No. 2003085024, U.S. Pat. Application No. 2004089442, U.S. Pat. No. 6,991,024, U.S. Pat. Application No. 2003062149, U.S. Pat. No. 6,942,018, U.S. Pat. Application No. 2003068229, U.S. Pat. No. 6,619,925, U.S. Pat. Application No. 2003205582, U.S. Pat. Application No. 2004138588, U.S. Pat. Application No. 2005235732, U.S. Pat. Application No. 2005238503, U.S. Pat. Application No. 2005248606, U.S. Pat. No. 6,916,159, U.S. Pat. Application No. 2004120827, U.S. Pat. Application No. 2004147907, U.S. Pat. Application No. 2005126912, U.S. Pat. Application No. 2005055014,and PCT publication Nos. WO 2004070085, WO 2004069390, WO 2005016558 WO 9615576, WO 9916162, WO 9901663, WO 2000055502, WO 9723178, WO 2000055502, WO 2000054745, WO 2001031322, WO 2002095341, WO 2003029731, WO 2003028862, WO 2003028861, WO 2002094440, WO 2002069935, WO 2004036136, WO 2001061314, WO 2003092662, WO 9943383, WO 20044032994, WO 2004061958,U.S. Pat. Application No. 20060116663, U.S. Pat. Application No.20040147907, U.S. Pat. Application No. 20030205582, U.S. Pat. Application No. 20060052768, U.S. Pat. Application No. 20060041229, and U.S. Pat. Application No. 2006/0116641, the entire contents of each of which are incorporated by reference.

What is claimed is:

1. A fluid delivery apparatus comprising:
a first osmotic compartment coupled to a stationary semi-permeable membrane, wherein:
the stationary semi-permeable membrane permits fluid migration across the membrane and into the first osmotic compartment; and
the first osmotic compartment is adapted to initially include a chemical composition that functions to alter osmotic pressure within the first osmotic compartment upon fluid migration across the stationary semi-permeable membrane;
a second osmotic compartment coupled to a portion of the stationary semi-permeable membrane, wherein the second osmotic compartment contains a fluid capable of migrating from the second osmotic compartment across the stationary semi-permeable membrane into the first osmotic compartment;
a displaceable barrier member coupled to the first osmotic compartment, wherein the displaceable barrier member is displaced in response to alterations in osmotic pressure within the first osmotic compartment;
a medication reservoir including a fluid outlet for delivering a fluid medication from the medication reservoir, wherein the medication reservoir is coupled to the displaceable barrier member such that a fluid medication is delivered from the medication reservoir through the fluid outlet upon displacement of the displaceable barrier member;
a solute reservoir including a fluid conduit that is capable of delivering a solute fluid from the solute reservoir into the first osmotic compartment, wherein delivery of the solute fluid into the first osmotic compartment functions to alter osmotic pressure within the first osmotic compartment;
a solute delivery system that delivers the solute fluid from the solute reservoir into the first osmotic compartment; and
a solute delivery controller that controls delivery of the solute fluid from the solute reservoir into the first osmotic compartment.

2. The fluid delivery apparatus of claim 1, wherein the apparatus is implantable within an individual.

3. The fluid delivery apparatus of claim 2, wherein the stationary semi-permeable membrane is exposed to a body fluid of the individual and the apparatus uses water in a body fluid of the individual to modulate osmotic pressure within the apparatus as the water migrates across the stationary semi-permeable membrane into the first osmotic compartment.

4. The fluid delivery apparatus of claim 1, wherein a portion of the stationary semi-permeable membrane is disposed on the apparatus to be exposed to a fluid in an external environment, such that the fluid in the external environment can migrate across the stationary semi-permeable membrane into the first osmotic compartment.

5. The fluid delivery apparatus of claim 1, wherein the medication reservoir contains a fluid medication selected from the group consisting of an antibiotic agent, an antiviral agent, a chemotherapeutic agent, an anti-inflammatory agent, or combinations thereof.

6. The fluid delivery apparatus of claim 1, further comprising a second displaceable barrier member coupled to the second osmotic compartment.

7. The fluid delivery apparatus of claim 1, wherein the first osmotic compartment includes a first electrode and the second osmotic compartment includes a second electrode so as to form an electrochemical cell,
wherein the first and second osmotic compartments include a fluid electrolyte in communication with the first and second electrodes and further wherein the first and second electrodes are coupled to a controller that controls an electrical signal sent to or received from the first or second electrodes.

8. The fluid delivery apparatus of claim 1, wherein the solute reservoir further includes a fluid conduit capable of delivering a solute fluid from the solute reservoir into the second osmotic compartment, wherein delivery of the solute fluid into the second osmotic compartment modulates the osmotic pressure within the first osmotic compartment, the second osmotic compartment or the first osmotic compartment and the second osmotic compartment.

9. The fluid delivery apparatus of claim 8, wherein fluid conduit capable of delivering a solute fluid from the solute reservoir into the second osmotic compartment and/or the fluid conduit capable of delivering a solute fluid from the solute reservoir into the first osmotic compartment comprises a valve to direct or meter fluid flow.

10. The fluid delivery apparatus of claim 1, wherein the first osmotic compartment, the second osmotic compartment or the first osmotic compartment and the second osmotic compartment comprise a fluid bleed member that can modulate the fluid volume in the first osmotic compartment, the second osmotic compartment or the first osmotic compartment and the second osmotic compartment.

11. The fluid delivery apparatus of claim 10, wherein a fluid bleed member comprises a valve to direct or meter fluid flow.

12. The fluid delivery apparatus of claim 2, wherein the apparatus uses body fluids within the individual as a water supply that functions to modulate osmotic pressure within the apparatus.

13. The fluid delivery apparatus of claim 2, wherein operation of the apparatus produces ions that are released into the body of the individual.

14. The fluid delivery apparatus of claim 2, wherein operation of the apparatus produces ions that are released into a moveable or deformable trap member so that the ions are not released into the body of the individual.

15. The fluid delivery apparatus of claim 14, wherein the moveable or deformable trap member is coupled to the medication reservoir such that fluid medication is delivered from the medication reservoir through the fluid outlet upon displacement of the moveable or deformable trap member.

16. The fluid delivery apparatus of claim 14, wherein the moveable or deformable trap member comprises a piston, a bellows, a bladder, a diaphragm, a plunger or a balloon or combinations thereof.

17. The fluid delivery apparatus of claim 7, wherein the first osmotic compartment or the second osmotic compartment comprises a chemical reagent which expands upon a chemical and/or electrochemical reaction.

18. A method of modulating fluid medication delivery from a medication reservoir within a fluid medication delivery apparatus, wherein the apparatus comprises:
a first osmotic compartment coupled to a stationary semi-permeable membrane, wherein:
the stationary semi-permeable membrane permits fluid migration across the membrane and into the first osmotic compartment; and
the first osmotic compartment is adapted to initially include a chemical composition that functions to alter osmotic pressure within the first osmotic compartment upon fluid migration across the stationary semi-permeable membrane;
a second osmotic compartment coupled to a portion of the stationary semi-permeable membrane, wherein the second osmotic compartment contains a fluid capable of migrating from the second osmotic compartment across the stationary semi-permeable membrane into the first osmotic compartment;
a displaceable barrier member coupled to the first osmotic compartment, wherein the displaceable barrier member is displaced in response to alterations in osmotic pressure within the first osmotic compartment;
a medication reservoir including a fluid outlet for delivering a fluid medication from the medication reservoir, wherein the medication reservoir is coupled to the displaceable barrier member such that fluid medication is delivered from the medication reservoir through the fluid outlet upon displacement of the displaceable barrier member;
a solute reservoir including a fluid conduit that is capable of delivering a solute fluid from the solute reservoir into the first osmotic compartment, wherein delivery of the solute fluid into the first osmotic compartment functions to alter osmotic pressure within the first osmotic compartment;
a fluid delivery system that delivers the solute fluid from the solute reservoir into the first osmotic compartment; and
a solute delivery controller that controls delivery of the solute fluid from the solute reservoir into the first osmotic compartment;
the method comprising:
delivering an amount of the solute fluid from the solute reservoir into the first osmotic compartment, wherein the amount of fluid delivered from the solute reservoir into the first osmotic compartment is sufficient to alter the osmotic pressure within the first osmotic compartment so as to displace the displaceable barrier member and modulate delivery of the fluid medication from the medication reservoir through the fluid outlet.

19. The method of claim 18, wherein the amount of the solute fluid delivered from the solute reservoir into the first osmotic compartment is sufficient to produce an oscillating fluid delivery profile.

20. The method of claim 18, wherein the apparatus further comprises a fluid medication disposed in the medication reservoir and the amount of the solute fluid delivered from the solute reservoir into the first osmotic compartment is sufficient to produce a fluid medication delivery profile comprising a first amount of fluid medication delivered within hours 1-10 after initiating fluid delivery and a second amount of fluid medication delivered within hours 11-20 after initiating fluid delivery, wherein the first amount of fluid medication delivered within hours 1-10 is at least 2, 3, 4, 5, 7, 8 or 9 times the second amount of fluid medication delivered within hours 11-20.
